(19) United States
(12) Patent Application Publication
(10) Pub. No.: US 2013/0042918 A1
(43) Pub. Date: Feb. 21, 2013

(54) OLIGOTHIOPHENES

(71) Applicants: Richard Evans, Victoria (AU); Akhil Gupta, Victoria (AU); Abdelselam Saeed Ali, Victoria (AU)

(72) Inventors: Richard Evans, Victoria (AU); Akhil Gupta, Victoria (AU); Abdelselam Saeed Ali, Victoria (AU)

(73) Assignee: COMMONWEALTH SCIENTIFIC AND INDUSTRIAL RESEARIAL ORGANISATION

(21) Appl. No.: 13/695,740

(22) PCT Filed: May 5, 2011
(86) PCT No.: PCT/AU2011/000514

§ 371 Date: Nov. 1, 2012

(30) Foreign Application Priority Data
May 5, 2010 (AU) 2010901925

(51) Int. Cl. H01L 51/46; C07D 495/04; C07D 495/14; C07D 409/14

(52) U.S. Cl. 136/263; 546/280.4; 546/281.1; 546/276.7

(57) ABSTRACT

Compounds of Formula (I):

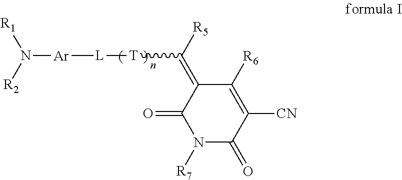

Wherein:
R₁ and R₂ are independently selected from the group consisting of optionally substituted C₁-C₂₀ alkyl, optionally substituted C₃-C₈ cycloalkyl, optionally substituted aromatic, and optionally substituted heteroaromatic groups or R₁ and R₂ together with the nitrogen atom to which they are attached comprise an optionally substituted saturated or unsaturated ring which may optionally contain further heteroatoms selected from the group consisting of O, N and S, and may optionally be further fused to one or more other rings;
Ar is selected from the group consisting of optionally substituted aromatic and optionally substituted heteroaromatic groups;
L is a linker which is a direct bond or is selected from the group consisting of optionally substituted C₂ alkenylene and optionally substituted C₂ alkynylene;
T is independently selected from the group consisting of:

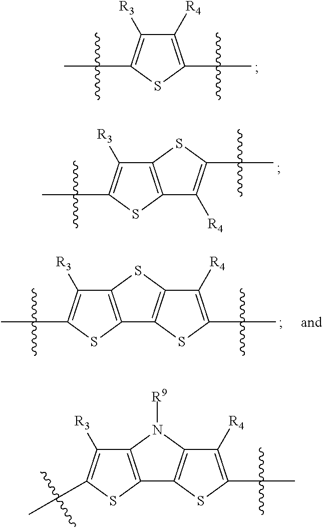

R₃, R₄ and R₉ are independently selected from the group consisting of hydrogen, optionally substituted C₁-C₁₀ alkyl, optionally substituted C₃-C₈ cycloalkyl and optionally substituted C₁-C₁₀ alkoxy groups, or a pair of groups selected from R₃, R₄ and R₉ may together with the carbon atoms to which they are attached comprise an optionally substituted saturated or unsaturated ring which may optionally contain one or more heteroatoms selected from the group consisting of O, N and S, and may optionally be further fused to one or more other rings;
R₅ is selected from the group consisting of hydrogen, optionally substituted C₁-C₈ alkyl, optionally substituted C₃-C₈ cycloalkyl, and optionally substituted aromatic groups;
R₆ is selected from the group consisting of optionally substituted C₁-C₈ alkyl, optionally substituted C₁-C₈ perfluorinated alkyl, optionally substituted C₃-C₈ cycloalkyl, optionally substituted aromatic, and optionally substituted heteroaromatic groups;
R₇ is selected from the group consisting of optionally substituted C₁-C₃₀ alkyl wherein one or more carbon atoms of the alkyl are optionally replaced with one or more of O, S, NR₈, carbonyl or thiocarbonyl; optionally substituted C₃-C₈ cycloalkyl; optionally substituted C₂-C₁₂ alkenyl wherein one or more carbon atoms of the alkenyl are optionally replaced with one or more of O, S, NR₈, carbonyl or thiocarbonyl; optionally substituted C₂-C₈ alkynyl wherein one or more carbon atoms of the alkynyl are optionally replaced with one or more of O, S, NR₈, carbonyl or thiocarbonyl; optionally substituted C₃-C₁₂alkoxy; optionally substituted aromatic;
and optionally substituted heteroaromatic groups; wherein R₈ is hydrogen or R₆, and
n is an integer of 1 to 10.

The compounds are capable of charge transportation and have application in organic photovoltaic devices such as dye sensitised solar cells.

### FIELD

The present application relates to new chemical compounds useful in organic photovoltaic applications, and to photovoltaic devices including solar cells and dye sensitised solar cells and photodetectors.

### BACKGROUND

Photovoltaic devices include heterojunction and bilayer organic photovoltaic cells, sometimes referred to as organic photovoltaics (OPVs), hybrid solar cells and dye sensitised solar cells, which are also known as Gratzel cells.

Photovoltaic devices contain a combination of electron acceptor materials and electron donor materials (or hole accepting materials) in the active layer. Absorption of a photon results in the generation of a weakly-bound electron-hole pair (or exciton) in the active layer. Dissociation of the bound electron-hole pair is facilitated by the interface between the electron donor and electron acceptor materials. The separated holes and electrons travel towards respective electrodes and consequently generate a voltage potential at the electrodes.

Poly 3-hexylthiophene is an example of a polymeric organic material used as an electron donor material in photovoltaic devices, together with fullerene as an example of an electron acceptor material. The two materials may be present as layers, forming a bilayer photovoltaic cell, or may be present as a blend, forming a bulk heterojunction photovoltaic cell. In bulk heterojunction photovoltaic cells the donor material (or p-type conductor) and acceptor material (n-type conductor) are presented in a tight blend in the active (specifically, photoactive) layer of a device, and the concentration of each component often gradually increases when approaching the corresponding electrode. This provides an increase in the total surface area of the junctions between the materials and facilitates the exciton's dissociation. In organic solar cells the electron donor and acceptor materials are both organic materials. In hybrid solar cells, one type of which is a dye sensitised solar cell, one material is typically an inorganic material and the other is an organic material. In dye sensitised solar cells, dye materials, also known as “sensitisers” or charge transporting chromophores, are used as a charge generating material, typically with an inorganic semiconductor. One example of this is the use of electron donor dyes with an n-type semi conductor such as titania, as the electron acceptor material.

There has been an emerging trend to develop new chemical compounds capable of charge transportation (as either the electron donor or electron acceptor material) for use in organic photovoltaic applications.

In charge transportation materials recently developed for such applications, the trend has been towards the use of compounds containing a donor electron group (such as an N,N-diarylamino group) at one end, a combination of an oligothiophene and an acceptor electron group at the other end, and a highly aromatic linker based on a pi system, such as phenyl, linking the two ends.

Conventional push-pull dye structures make use of a thiophene (or oligothiophene) unit as a π-electron bridge between an aryl amine (an electron donor) and a dicyanovinylidene (an electron acceptor). The acceptor is usually dicyanovinylidene (═C(CN₂)) for bulk heterojunctional (BHJ) devices and carboxylcyanovinylidene for dye sensitised solar cells (DSSC). Use of an aromatizable acceptor in DSSC such as Rodanine acetic acid has also been reported.

There is a need for further chemical compounds that can be used in such applications, which may provide improved charge delocalization in the compound. There is also a need for devices containing these new compounds.

### SUMMARY

In a first aspect there is provided a compound of formula I:

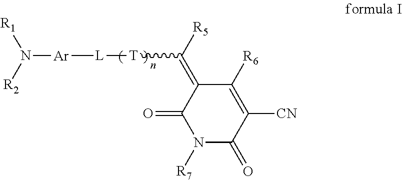

wherein:

R₁ and R₂ are independently selected from the group consisting of optionally substituted C₁-C₂₀ alkyl, optionally substituted C₃-C₈ cycloalkyl, optionally substituted aromatic, and optionally substituted heteroaromatic groups or R₁ and R₂ together with the nitrogen atom to which they are attached comprise an optionally substituted saturated or unsaturated ring which may optionally contain further heteroatoms selected from the group consisting of O, N and S, and may optionally be further fused to one or more other rings;

Ar is selected from the group consisting of phenyl, fluorenyl, dialkylfluoroenyl and thiophenyl;

L is a linker which is a direct bond or is selected from the group consisting of optionally substituted C₂ alkenylene and C₂ alkynylene;

T is independently selected from the group consisting of:

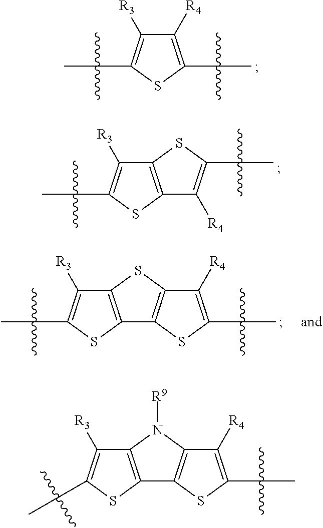

R₃, R₄ and R₉ are independently selected from the group consisting of hydrogen, optionally substituted C₁-C₁₀ alkyl, optionally substituted C₃-C₈ cycloalkyl and optionally substituted C₁-C₁₀ alkoxy groups, or a pair of groups selected from R₃, R₄ and R₉ may together with the carbon atoms to which they are attached comprise an optionally substituted saturated or unsaturated ring which may optionally contain one or more heteroatoms selected from the group consisting of O, N and S, and may optionally be further fused to one or more other rings;

R₅ is selected from the group consisting of hydrogen, optionally substituted C₁-C₈ alkyl, optionally substituted C₃-C₈ cycloalkyl, and optionally substituted aromatic groups;

R₆ is selected from the group consisting of optionally substituted C₁-C₈ alkyl, optionally substituted C₁-C₈ perfluorinated alkyl, optionally substituted C₃-C₈ cycloalkyl, optionally substituted aromatic, and optionally substituted heteroaromatic groups;

R₇ is selected from the group consisting of optionally substituted C₁-C₃₀ alkyl wherein one or more carbon atoms of the alkyl are optionally replaced with one or more of O, S, NR₈, carbonyl or thiocarbonyl; optionally substituted C₃-C₈ cycloalkyl; optionally substituted C₂-C₁₂ alkenyl wherein one or more carbon atoms of the alkenyl are optionally replaced with one or more of O, S, NR₈, carbonyl or thiocarbonyl; optionally substituted C₂-C₈ alkynyl wherein one or more carbon atoms of the alkynyl are optionally replaced with one or more of O, S, NR₈, carbonyl or thiocarbonyl; optionally substituted C₃-C₁₂ alkoxy; optionally substituted aromatic; and optionally substituted heteroaromatic groups; wherein R₈ is hydrogen or R₆, and

n is an integer of 1 to 10; with the proviso that when T is

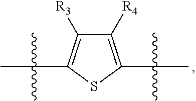

n is an integer of 2 to 10 and Ar is selected from phenyl, fluorenyl and dialkylfluorenyl.

In a second aspect there is provided a photovoltaic device comprising:
a first electrode,
a second electrode, and
an active material in electrical contact with the first and second electrodes, the active material comprising a compound of formula I as defined above and a second material which is a charge accepting material,
   wherein the device generates an electrical potential upon the absorption of photons.

In one embodiment, the device is a dye sensitised solar cell comprising:
an anode,
a cathode,
a charge accepting material on one electrode,
a compound of formula I, as defined above, in contact with the charge accepting material, and
a charge transport material in contact with the compound of formula I and the other electrode.

In a third aspect there is provided a process for the preparation of a compound of formula I comprising reacting compound C:

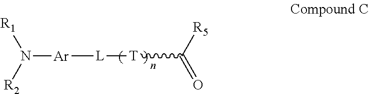

wherein R₁, R₂, R₅, Ar, L, T and n are as defined in formula I above, with compound D:

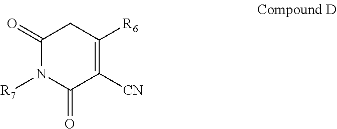

wherein R₆ and R₇ are as defined in formula I above.

Preferred details of the compound, process and the device are set out in the detailed description below.

### BRIEF DESCRIPTION OF THE FIGURES

FIG. 1 is a schematic illustration of a photovoltaic device, in the form of a bilayer photovoltaic cell, according to one embodiment of the invention.

FIG. 2 is a schematic illustration of a photovoltaic device, in the form of a bulk heterojunction photovoltaic cell, according to a second embodiment of the invention.

FIG. 3 is a schematic illustration of a photovoltaic device, in the form of a dye sensitised solar cell, according to a third embodiment of the invention.

FIG. 4 is an I-V curve or graph of voltage vs current density for a photovoltaic device according to one embodiment of the invention incorporating Compound Example 2.

FIG. 5 is an I-V curve or graph of voltage vs current density for a photovoltaic device according to another embodiment of the invention incorporating Compound Example 2.

FIG. 6 is an I-V curve or graph of voltage vs current density for a photovoltaic device according to another embodiment of the invention incorporating Compound Example 3.

FIG. 7 is an I-V curve or graph of voltage vs current density for a photovoltaic device according to another embodiment of the invention incorporating Compound Example 4.

FIG. 8 is an I-V curve or graph of voltage vs current density for a photovoltaic device according to another embodiment of the invention incorporating Compound Example 5.p

FIG. 9 is an I-V curve or graph of voltage vs current density for a photovoltaic device according to another embodiment of the invention incorporating Compound Example 6.

### DETAILED DESCRIPTION

The present invention relates to new compounds, processes for preparing the compounds, and their use in photovoltaic devices. It is noted that the term “device” is used broadly to refer to any device containing the stated electrodes and active material, and thus encompasses solar cells, photodetectors and the like.

The compounds of the present application are based on a donor-acceptor design which has greater absorption of visible light than current oligothiophene-based materials due to the highly efficient electron donor-acceptor configuration of the substituents on a thiophene (or oligothiophene) core.

The structure includes a direct link between the thiophene (or oligothiophene) unit and a strongly electron withdrawing cyanopyridone aromatizable acceptor group. The thiophene (or oligothiophene) unit is linked directly or indirectly to a highly aromatic group which is linked directly to an amino electron donor group.

The use of cyanopyridone aromatizable acceptor groups in push-pull dyes and photovoltaic cells is new.

When a cyanopyridone aromatizable acceptor group is connected to an amino electron donor group via an aromatic group, the acceptor and donor groups produce a synergistic effect which causes a large red shifting of the absorbance maxima. Photovoltaic devices containing such compounds will benefit from these properties.

The compounds of the invention may be referred to as oligothiophene compounds.

In formula I, n is an integer of 1 to 10. According to some embodiments, n is an integer of 1 to 6.

R₁ and R₂ are independently selected from the group consisting of optionally substituted C₁-C₂₀ alkyl, optionally substituted C₃-C₈ cycloalkyl, optionally substituted aromatic and optionally substituted heteroaromatic groups.

The term “alkyl group” encompasses straight chained or branched alkyl groups of C₁ to C₃₀, and encompasses groups of the formula —CₓH₂ₓ₊₁, where x is an integer of 1 to 30, such as an integer of 1 to 20, or an integer of 1 to 10, or an integer of 1 to 8, or an integer of 1 to 6. Examples include methyl, ethyl, propyl, hexyl, iso-butyl, tert-butyl, and so forth. Unless the context requires otherwise, alkyl also encompasses alkyl groups containing one less hydrogen atom, such that the group is attached via two positions. Such groups are also referred to as “alkylene” groups.

The term “cycloalkyl group” refers to non-aromatic cyclic hydrocarbon groups having from 3 to 8 carbon atoms. Examples include cyclopropyl, cyclopentyl and cyclohexyl.

The term “aromatic group” or “aryl” refers to any group containing an aromatic ring system. Such groups may contain fused ring systems (such as napthyl and fluorenyl), linked ring systems (such as biphenyl groups), and may be substituted or unsubstituted. Any substituents that do not adversely impact on the electronic properties of the ring system are permissible, and suitable examples include one or more substituents selected from C₁-C₂₀ alkyl, C₁-C₁₀ alkoxy, hydroxyl, carbonyl, carboxylic acid, halo, aryl, thio-C₁-C₁₀alkyl, cyano, halo-C₁-C₁₀alkyl such as perfluorinated C₁-C₁₀alkyl, dialkylamino, diarylamine, N-carbazol, heteroaryl, biphenyl, silyl, trimethylsilyl, silyl ether, methacryloxy, acryloxy, hydroxyalkyleneoxy and 2-bromo-2-methylpropanoate. Halo refers to a halogen such as F, Cl, Br or I. Halo-C₁-C₁₀alkyl refers to a C₁-C₁₀alkyl substituted with one or more halogen. Thio-C₁-C₁₀alkyl is the thio (S-containing) equivalent of alkoxy. Carbonyl encompasses carboxylic acids, esters aldehydes and ketones.

The term “heteroaromatic group” or similarly “heteroaryl” refers to any group containing a heteroaromatic ring system. The heteroatoms in the heteroaromatic group may be selected from one or more of O, N and S. Such groups may be substituted or unsubstituted (such as substituted or unsubstituted pyridyl, thienyl, furyl, indolinyl and so forth), and may contain fused ring systems (such as purines), including a fused heteroaromatic and carbon-based aromatic rings (such as benzothiophenyl, indolyl, isoindolyl, benzofuranyl, isobenzofuranyl, benzimidazolyl, indazolyl, benzoxazolyl, benzisoxazolyl, benzothiazolyl, quinolinyl, isoquinolinyl, quinoxalinyl, quinazolinyl, and cinnolinyl), and linked ring systems (such as oligothiophene or polypyrrole). Suitable substituents are the same as those listed above for the aromatic group.

R₁ and R₂ may, together with the nitrogen atom to which they are attached, comprise an optionally substituted saturated or unsaturated ring which may optionally contain further heteroatoms and may optionally be further fused to one or more other rings.

In some embodiments, the saturated or unsaturated ring may be an optionally substituted 5-7 membered ring. In some embodiments, the optionally substituted saturated or unsaturated ring contains at least one further heteroatom selected from the group consisting of O, N and S.

Suitable saturated rings include pyrrolidinyl, imidazolidinyl, pyrazolidinyl, piperidinyl, morpholinyl, thiomorpholinyl and piperazinyl. Suitable unsaturated rings include pyrrolyl, pyrrolinyl, pyrazolyl, pyrazolinyl and triazolyl. Suitable substituents are the same as those listed above for the aromatic group.

The saturated or unsaturated ring may optionally be further fused to one or more other rings. The one or more other rings may be an optionally substituted 5-7 membered saturated or unsaturated ring. In some embodiments, the other ring is a benzene ring. Suitable fused ring systems include indolyl, isoindolyl, indolinyl, indazolyl, benzimidazolyl, purinyl, carbazole, carbolinyl, benzazepinyl and benzodiazepinyl. Suitable substituents are the same as those listed above for the aromatic group.

According to some embodiments, R₁ and R₂ are independently selected from the group consisting of phenyl, substituted phenyl, fluorenyl, and substituted fluorenyl. Suitable substituents are the same as those listed above for the aromatic group.

Ar is selected from the group consisting of optionally substituted aromatic and optionally substituted heteroaromatic groups. Aromatic and heteroaromatic groups are as defined above.

According to some embodiments, Ar is phenyl, fluorenyl, dialkylfluoroenyl or thiophenyl.

According to some embodiments, Ar is an optionally substituted fused aromatic (such as napthyl and fluorenyl) or an optionally substituted linked aromatic group (such as a biphenyl group). Suitable substituents are the same as those listed above for the aromatic group.

According to some embodiments, Ar is an optionally substituted 5- or 6-membered heteroaromatic group containing at least one of O, N and S, fused to a benzene ring. Suitable groups include benzothiophenyl, indolyl, isoindolyl, benzofuranyl, isobenzofuranyl, benzimidazolyl, indazolyl, benzoxazolyl, benzisoxazolyl, benothiazolyl, quinolinyl, isoquinolinyl, quinoxalinyl, quinazolinyl, and cinnolinyl. Suitable substituents are the same as those listed above for the aromatic group.

L is a linker which is a direct bond or is selected from the group consisting of optionally substituted C₂ alkenylene and C₂ alkynylene. Suitable substituents are the same as those listed above for the aromatic group. In some embodiments L is a C₂ cyanoalkenylene. In some embodiments L is a direct bond.

The term “alkenyl group” refers to straight chain or branched hydrocarbon groups having at least one double bond of either E or Z stereochemistry where applicable and 2 to 18 carbon atoms. In some embodiments, the alkenyl group has 2 to 10 carbon atoms, or 2 to 8 carbon atoms or 2 to 6 carbon atoms. Examples include vinyl, 1-propenyl, 1- and 2-butenyl, hexenyl, butadienyl, hexadienyl, hexatrienyl and so forth. Unless the context requires otherwise, alkenyl also encompasses alkenyl groups containing one less hydrogen atom, such that the group is attached via two positions. Such groups are also referred to as “alkenylene” groups.

The term “alkynyl group” refers to straight chain or branched hydrocarbon groups having at least one triple bond and 2 to 18 carbon atoms. In some embodiments, the alkynyl group has 2 to 10 carbon atoms, or 2 to 8 carbon atoms or 2 to 6 carbon atoms. Examples include ethynyl, 1- or 2-propynyl, 2- or 3-butynyl and methyl-2-propynyl. Unless the context requires otherwise, alkynyl also encompasses alkynyl groups containing one less hydrogen atom, such that the group is attached via two positions. Such groups are also referred to as “alkynylene” groups.

T is independently selected from the group consisting of:

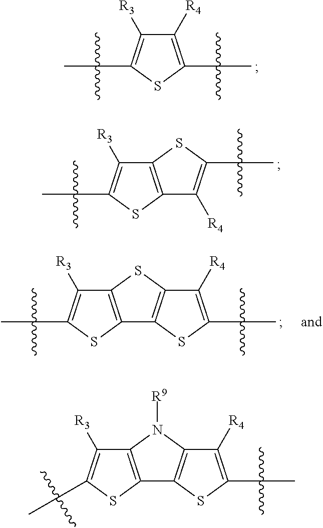

According to some embodiments, R₃, R₄ and R₉ are independently selected from the group consisting of hydrogen, optionally substituted C₁-C₁₀ alkyl, optionally substituted C₃-C₈ cycloalkyl, and optionally substituted C₁-C₁₀ alkoxy groups

Alkyl and cycloalkyl groups are as defined above. The term “alkoxy group” refers to the group —OCₓH₂ₓ₊₁, where x is an integer of 1 to 18, such as an integer of 1 to 10, or an integer of 1 to 8, or an integer of 1 to 6. Examples include methoxy, ethoxy, and so forth.

The oxygen atom may be located along the hydrocarbon chain, and need not be the atom linking the group to the remainder of the compound.

According to some embodiments R₃ is hydrogen. According to some embodiments R₄ is hydrogen. According to some embodiments, one of R₃ and R₄ is hydrogen, and the other of R₃ and R₄ is optionally substituted C₁-C₁₀ alkyl. According to some embodiments, R₃ and R₄ are both hydrogen.

According to some embodiments, R₉ is hydrogen. According to some embodiments R₉ is optionally substituted C₁-C₁₀alkyl. According to some embodiments, R₃ and R₄ are hydrogen and R₉ is optionally substituted C₁-C₁₀ alkyl.

A pair of groups selected from R₃, R₄ and R₉ (that is, R₃ and R₄ or R₃ and R₉, or R₄ and R₉) may, together with the carbon atoms to which they are attached, comprise an optionally substituted saturated or unsaturated ring which may optionally contain one or more heteroatoms selected from the group consisting of O, N and S, and may optionally be further fused to one or more other rings.

In some embodiments, the saturated or unsaturated ring may be an optionally substituted 5-7 membered ring. Suitable saturated rings include cycloalkyl, tetrahydrofuryl, tetrahydropyranyl, dioxolanyl, dioxanyl, pyrrolidinyl, imidazolidinyl, pyrazolidinyl, piperidinyl, piperazinyl, morpholinyl, thiomorpholinyl, dithainyl and trithianyl. Suitable unsaturated rings include phenyl, cycloalkenyl, furanyl, pyranyl, pyrrolyl, pyrrolinyl, pyrazolyl, pyrazolinyl, triazolyl, pyridinyl, pyridazinyl, pyrimidinyl, pyrazinyl, thiophenyl, thiazolyl and isothiazolyl. Suitable substituents are the same as those listed above for the aromatic group.

The saturated or unsaturated ring may optionally be further fused to one or more other rings. The one or more other rings may be an optionally substituted 5-7 membered saturated or unsaturated ring. In some embodiments, the other ring is a benzene ring. Suitable fused ring systems include napthyl, fluorenyl, indenyl, indolyl, isoindolyl, indolinyl, indazolyl, benzimidazolyl, purinyl, quinolinyl, isoquinolenyl, cinnolinyl, phtalazinyl, quinazolinyl, quinoxalinyl, naphthyridinyl, pteridinyl, carbazolyl, carbolinyl, benzazepinyl, benzodiazepinyl, benzofuranyl, benzothiophenyl and benzthiazolyl. Suitable substituents are the same as those listed above for the aromatic group.

R₅ is selected from the group consisting of hydrogen, optionally substituted C₁-C₈ alkyl, optionally substituted C₃-C₈ cycloalkyl, and optionally substituted aromatic groups. Alkyl, cycloalkyl and aromatic groups are as defined above. Suitable substituents are the same as those listed above for the aromatic group. According to some embodiments, R₅ is hydrogen.

R₆ is selected from the group consisting of optionally substituted C₁-C₈ alkyl, optionally substituted C₁-C₈ perfluorinated alkyl, optionally substituted C₃-C₆ cycloalkyl, optionally substituted aromatic, and optionally substituted heteroaromatic groups. The terms “alkyl group”, “cycloalkyl group”, “aromatic group” and “heteroaromatic group” are as defined above. In some embodiments, R₆ is an optionally substituted C₁-C₈ alkyl, an optionally substituted C₁-C₆ perfluorinated alkyl or an optionally substituted C₃-C₆ cycloalkyl. In some embodiments, R₆ is selected from the group consisting of methyl, ethyl and CF₃. As defined above, the term “alkyl group” encompasses straight chain or branched alkyl groups and in one embodiment R₆ is an optionally substituted branched C₂-C₈ alkyl. In some embodiments, R₆ is a thiophenyl group.

R₇ is selected from the group consisting of optionally substituted C₁-C₃₀ alkyl wherein one or more carbon atoms of the alkyl are optionally replaced with one or more of O, S, NR₈, carbonyl or thiocarbonyl; optionally substituted C₃-C₈ cycloalkyl; optionally substituted C₂-C₁₂ alkenyl wherein one or more carbon atoms of the alkenyl are optionally replaced with one or more of O, S, NR₈, carbonyl or thiocarbonyl; optionally substituted C₂-C₈ alkynyl wherein one or more carbon atoms of the alkynyl are optionally replaced with one or more of O, S, NR₈, carbonyl or thiocarbonyl; optionally substituted C₃-C₁₂ alkoxy; optionally substituted aromatic; and optionally substituted heteroaromatic groups; wherein R₈ is hydrogen or R₆. Suitable substituents are the same as those listed above for the aromatic group.

The terms “alkyl group”, “cycloalkyl group”, “alkenyl group”, “alkynyl group”, “alkoxy group”, “aromatic group” and “heteroaromatic group” are defined above.

In some embodiments, R₇ is selected from the group consisting of optionally substituted C₁-C₆ alkyl wherein the alkyl chain may be optionally interrupted with one or more of O, S, NR₈, carbonyl or thiocarbonyl; optionally substituted C₃-C₆ cycloalkyl; optionally substituted C₂-C₆ alkenyl wherein one or more carbon atoms of the alkenyl are optionally replaced with one or more of O, S, NR₈, carbonyl or thiocarbonyl; optionally substituted C₂-C₆ alkynyl wherein one or more carbon atoms of the alkynyl are optionally replaced with one or more of O, S, NR₈, carbonyl or thiocarbonyl; and optionally substituted C₃-C₆ alkoxy; wherein R₈ is hydrogen or R₆. In some embodiments, R₇ may be an optionally substituted branched C₂-C₁₀ alkyl wherein the alkyl chain may be optionally interrupted with one or more of O, S, NR₈, carbonyl or thiocarbonyl; optionally substituted branched C₂-C₁₀ alkenyl wherein one or more carbon atoms of the alkenyl are optionally replaced with one or more of O, S, NR₈, carbonyl or thiocarbonyl; optionally substituted branched C₃-C₁₀ alkynyl wherein one or more carbon atoms of the alkynyl are optionally replaced with one or more of O, S, NR₈, carbonyl or thiocarbonyl; or optionally substituted branched C₃-C₁₂ alkoxy; wherein R₈ is hydrogen or R₆.

In some embodiments, R₇ is an optionally substituted 5- or 6-heteroaromatic group containing one or more heteroatoms selected from the group consisting of O, N and S. Suitable substituents are the same as those listed above for the aromatic group.

In some embodiments, R₇ is an optionally substituted C₁-C₃₀ alkyl wherein the optional substituents are selected from the group consisting of hydroxyl, carboxylic acid, methacryloxy, acryloxy, hydroxyalkyleneoxy, 2-bromo-2-methylpropanoate, trimethylsilyl and silyl ether.

In some embodiments, R₇ is an aromatic group which is substituted with a carboxylic acid group.

In one embodiment there is provided compounds of formula I-A:

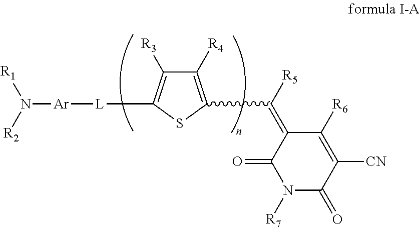

wherein R₁, R₂, R₃, R₄, R₅, R₆, R₇, Ar, L and n are as defined above.

Representative examples when n is 2 are as follows:

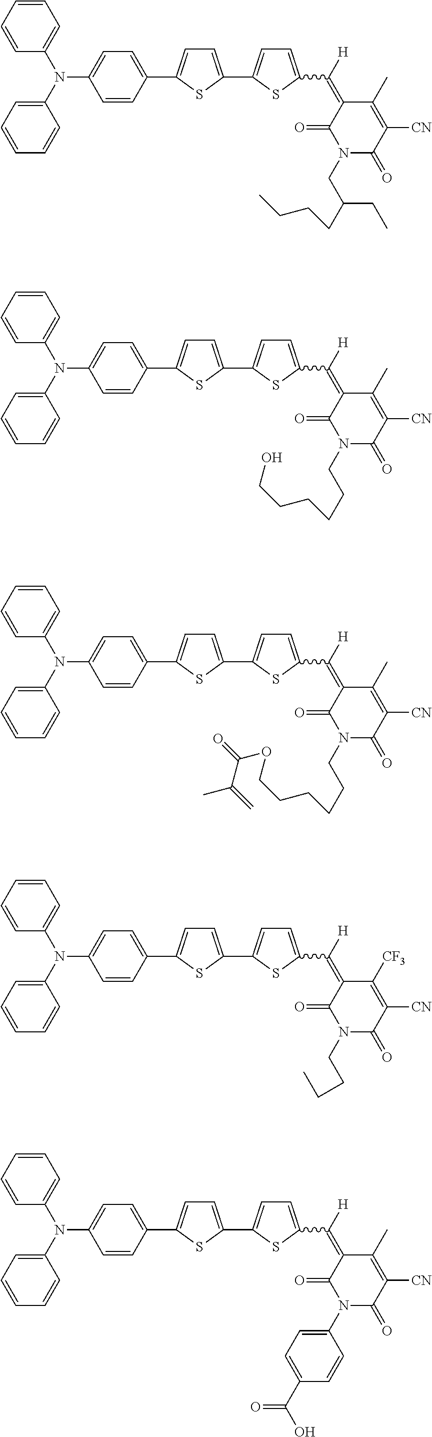 [SMILES: CCCCC(CC)CN1C(=O)C(=Cc2ccc(-c3ccc(-c4ccc(N(c5ccccc5)c5ccccc5)cc4)s3)s2)C(C)=C(C#N)C1=O ; CC1=C(C#N)C(=O)N(CCCCCCO)C(=O)C1=Cc1ccc(-c2ccc(-c3ccc(N(c4ccccc4)c4ccccc4)cc3)s2)s1 ; C=C(C)C(=O)OCCCCCCN1C(=O)C(=Cc2ccc(-c3ccc(-c4ccc(N(c5ccccc5)c5ccccc5)cc4)s3)s2)C(C)=C(C#N)C1=O ; CCCCN1C(=O)C(=Cc2ccc(-c3ccc(-c4ccc(N(c5ccccc5)c5ccccc5)cc4)s3)s2)C(C(F)(F)F)=C(C#N)C1=O ; CC1=C(C#N)C(=O)N(c2ccc(C(=O)O)cc2)C(=O)C1=Cc1ccc(-c2ccc(-c3ccc(N(c4ccccc4)c4ccccc4)cc3)s2)s1]

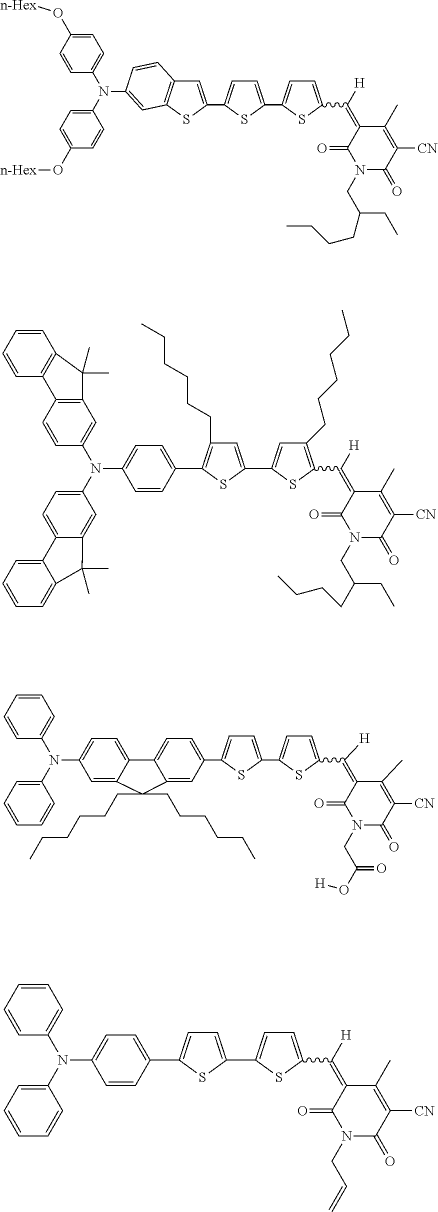

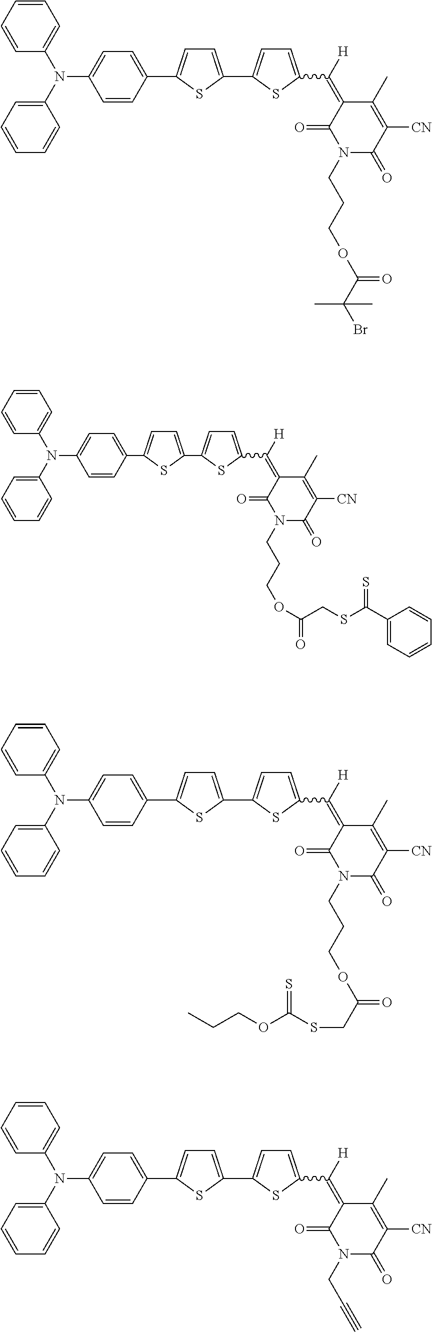 [SMILES: CC1=C(C#N)C(=O)N(CCCOC(=O)C(C)(C)Br)C(=O)C1=Cc1ccc(-c2ccc(-c3ccc(N(c4ccccc4)c4ccccc4)cc3)s2)s1 ; CC1=C(C#N)C(=O)N(CCCOC(=O)CSC(=S)c2ccccc2)C(=O)C1=Cc1ccc(-c2ccc(-c3ccc(N(c4ccccc4)c4ccccc4)cc3)s2)s1 ; CCCOC(=S)SCC(=O)OCCCN1C(=O)C(=Cc2ccc(-c3ccc(-c4ccc(N(c5ccccc5)c5ccccc5)cc4)s3)s2)C(C)=C(C#N)C1=O ; C#CCN1C(=O)C(=Cc2ccc(-c3ccc(-c4ccc(N(c5ccccc5)c5ccccc5)cc4)s3)s2)C(C)=C(C#N)C1=O]

Representative examples when n is 3 are as follows:

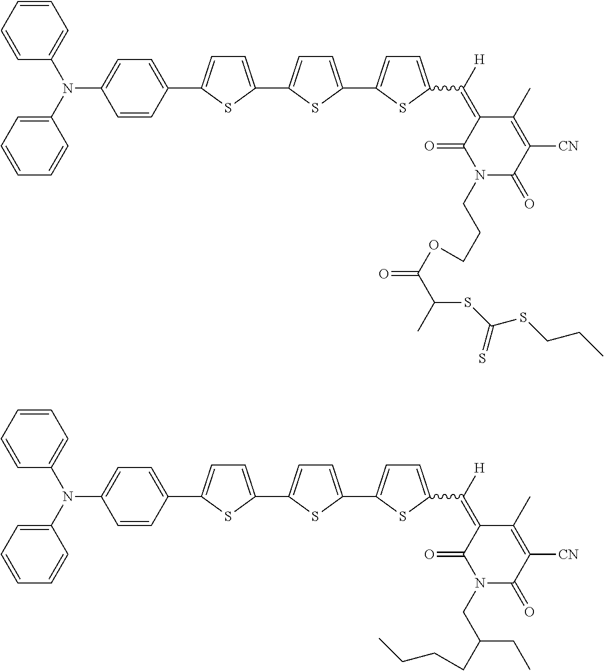 [SMILES: CCCSC(=S)SC(C)C(=O)OCCCN1C(=O)C(=Cc2ccc(-c3ccc(-c4ccc(-c5ccc(N(c6ccccc6)c6ccccc6)cc5)s4)s3)s2)C(C)=C(C#N)C1=O ; CCCCC(CC)CN1C(=O)C(=Cc2ccc(-c3ccc(-c4ccc(-c5ccc(N(c6ccccc6)c6ccccc6)cc5)s4)s3)s2)C(C)=C(C#N)C1=O]

Representative examples when n is 4 are as follows:

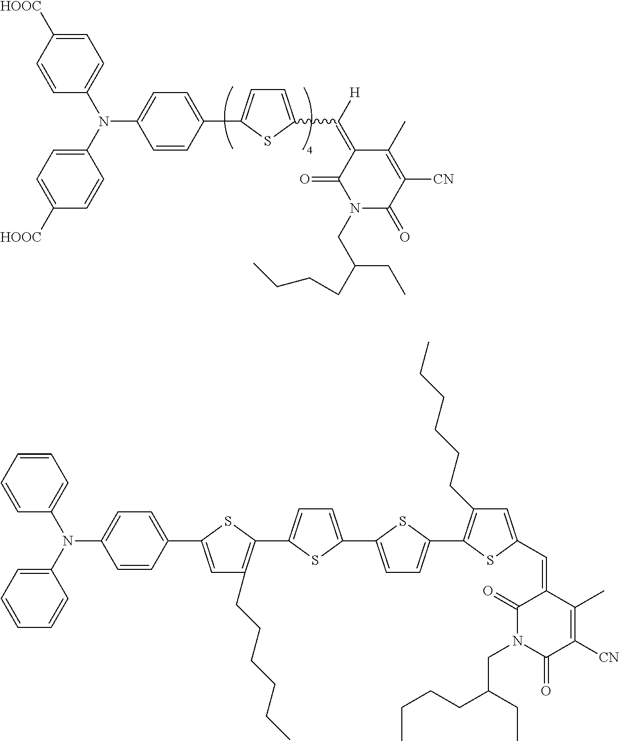

In another embodiment there is provided compounds of formula I-B:

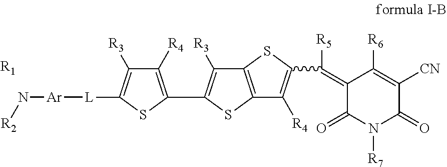

wherein R₁, R₂, R₃, R₄, R₅, R₆, R₇, Ar and L are as defined above.

Representative examples are as follows:

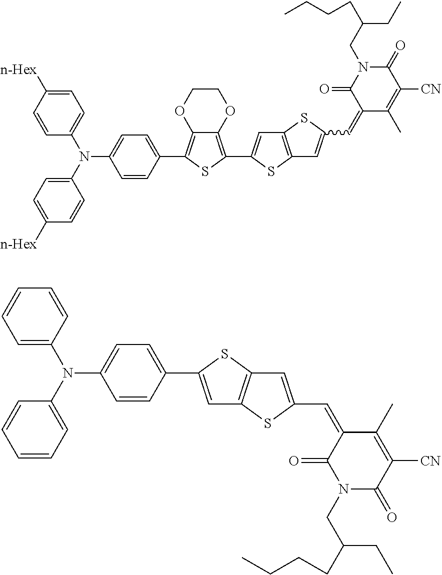

In another embodiment there is provided compounds of formula I-C:

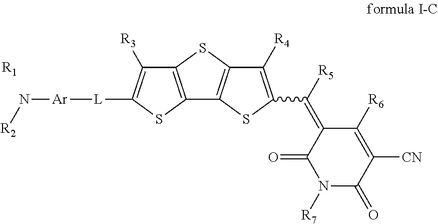

wherein R₁, R₂, R₃, R₄, R₅, R₆, R₇, Ar and L are as defined above.

A representative example is as follows:

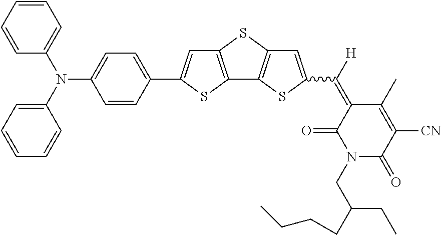 [SMILES: CCCCC(CC)CN1C(=O)C(=Cc2cc3sc4cc(-c5ccc(N(c6ccccc6)c6ccccc6)cc5)sc4c3s2)C(C)=C(C#N)C1=O]

In another embodiment there is provided compounds at formula I-D:

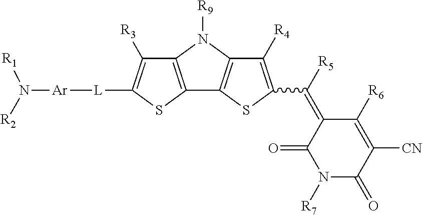

wherein R₁, R₂, R₃, R₄, R₅, R₆, R₇, R₉, Ar and L are as defined above.

A representative example is as follows:

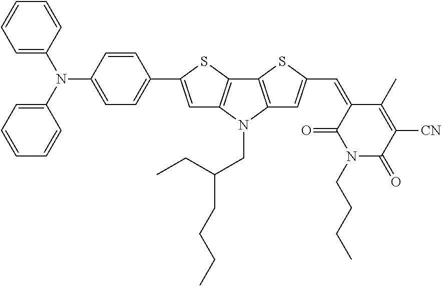 [SMILES: CCCCC(CC)Cn1c2cc(/C=C3\C(=O)N(CCCC)C(=O)C(C#N)=C3C)sc2c2sc(-c3ccc(N(c4ccccc4)c4ccccc4)cc3)cc21]

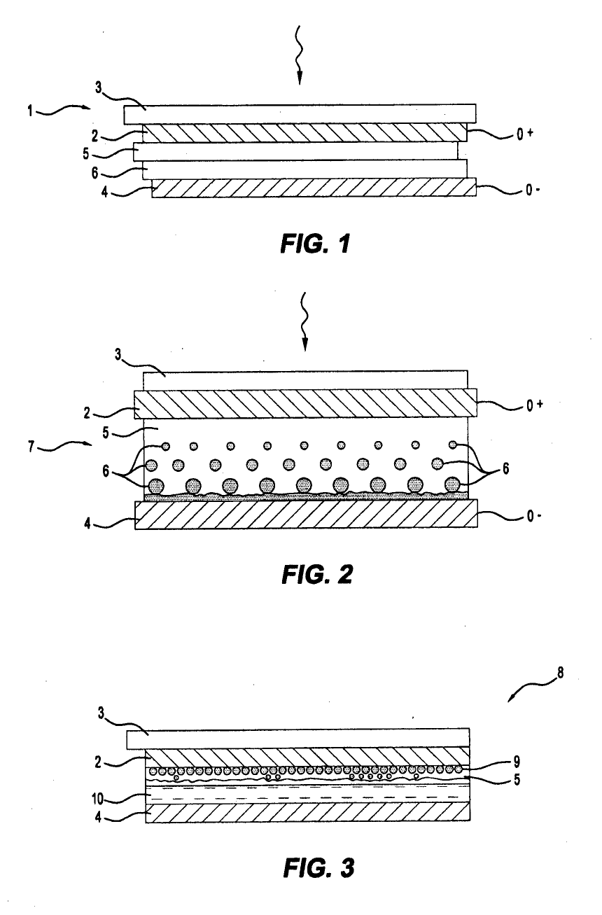

As indicated by the wavy line in formulae I, I-A, I-B and I-C, the compounds of the present application are not limited to any particular stereochemistry. The compounds may comprise mixtures of isomers in any ratio, racemic mixtures, a single isomer of the compound, or otherwise. The absence of a wavy line at a position corresponding to that shown in FIG. 1 in other parts of this specification should not be taken to imply specific stereochemistry about the double bond. The actual stereochemistry can only be determined by assessment of the compound as synthesised by the specified synthetic procedure.

Previously studied active materials for use in organic photovoltaic devices have included poly 3-hexyl thiophene (P3HT), which obtains its colour and function by an extended system. However, the number of thiophenes in the molecule to be used as an active material in organic photovoltaic devices can be reduced by induction of a dipole in the molecule. These molecules contain broad structural constituents of donor-aromatic linker-oligothiophene-acceptor. The aromatic groups have previously been highly aromatic groups such as benzene or fluorene, and the oligothiophene has been made of only 2-4 units.

In such systems, we have observed that there are strong inductive dipole generation in the materials, however resonance delocalization to give further absorption is not possible. We have explored and identified further features that provide improvements or useful alternatives to the known materials.

### Cyanopyridone Acceptor Groups

It has been found that the cyanopyridone group can become aromatized on quarternization of the amine nitrogen in the compound of formula I, such compounds provide considerable advantages. This phenomenon is illustrated below with respect to the groups thioparbituric acid, hydroxyphridone, phenyl isoxazolone and rhodamine. Rhodamine exhibits the weakest effect of the four illustrated.

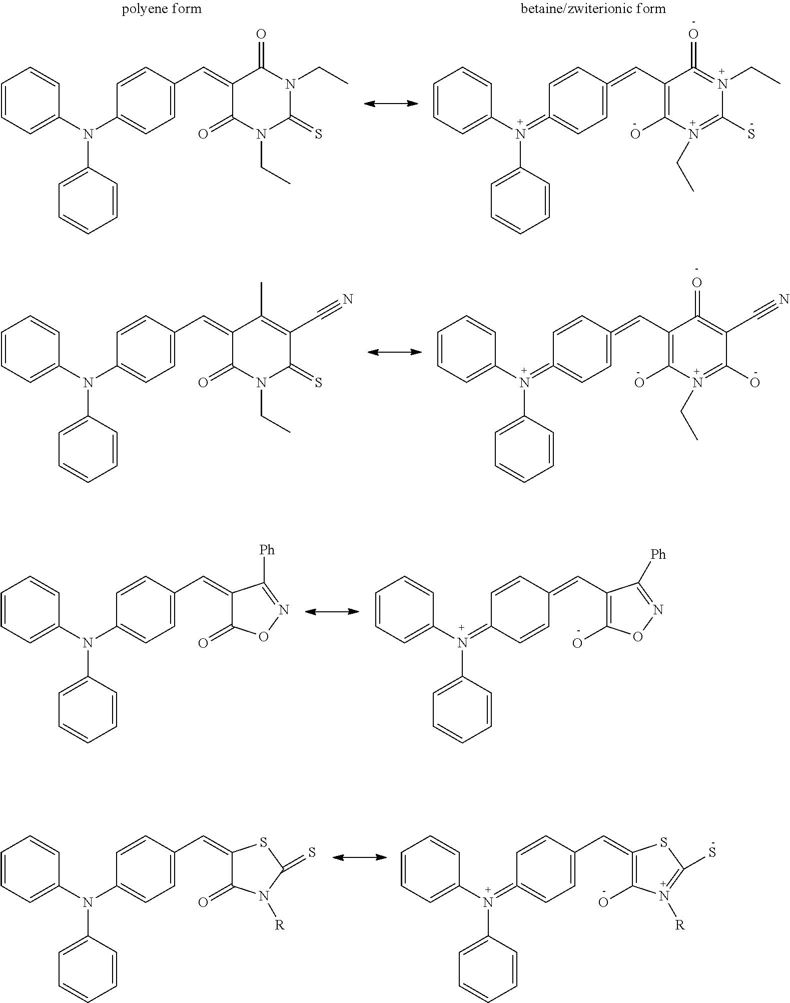

It is noted that the compounds in the illustration above are not themselves compounds of the present invention. However, they illustrate the same effect that applies to compounds of the present invention.

These aromatizable cyanopyridones allow the approach of a phenomenon known as the “cyanine” limit. Note that aromatization of the cyanopyridone is accompanied by the loss of aromaticity in the aromatic linker between the tertiary amine and acceptor. The cyanine limit is a state whereby the neutral polyene form and the canonical zwitterionic form contribute equally to the structure. This results in (1) the highest degree of conjucation, (2) high dipole moments and forced order hyperpolarizability, and (3) vanishing second order hyperpolarizability, and thus (4) no change in dipole moment on excitation.

This can be seen in x-ray structures where double bond alternation is reduced and sometimes even the partial zwitterionic forms are observed in the polar environment of a crystal. The structure below illustrates a molecule at the cyanine limit with the disappearance of double bond alternation.

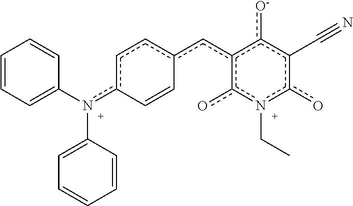

Other groups disclosed in the art offer minimal canonical forms and such molecules cannot approach the cyanine limit.

### Synthesis of Compounds

Examples demonstrating the synthesis of compounds of a full range of embodiments of the invention are set out in the Example section.

Generally, the synthesis involves:
reacting compound A with compound B to form compound C as follows:

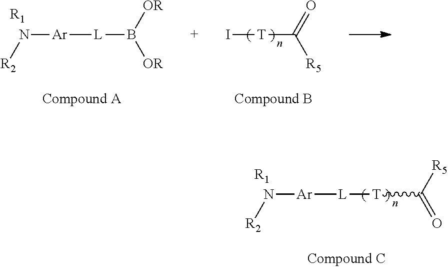

wherein R₁, R₂, R₅, T, Ar, L and n are as herein defined and R is hydrogen or an ester; and
reacting compound C with compound D as follows:

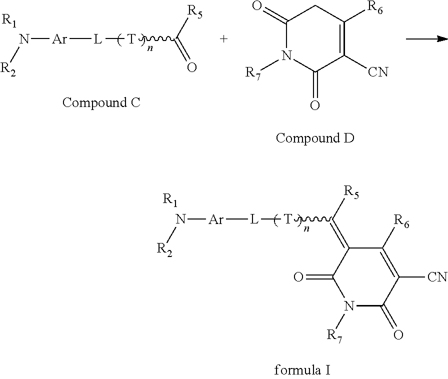

Suitable compounds of type A are available for purchase, or can be synthesised by techniques known in the art.

Suitable compounds of type B of the desired length n, can be synthesised according to the following scheme:

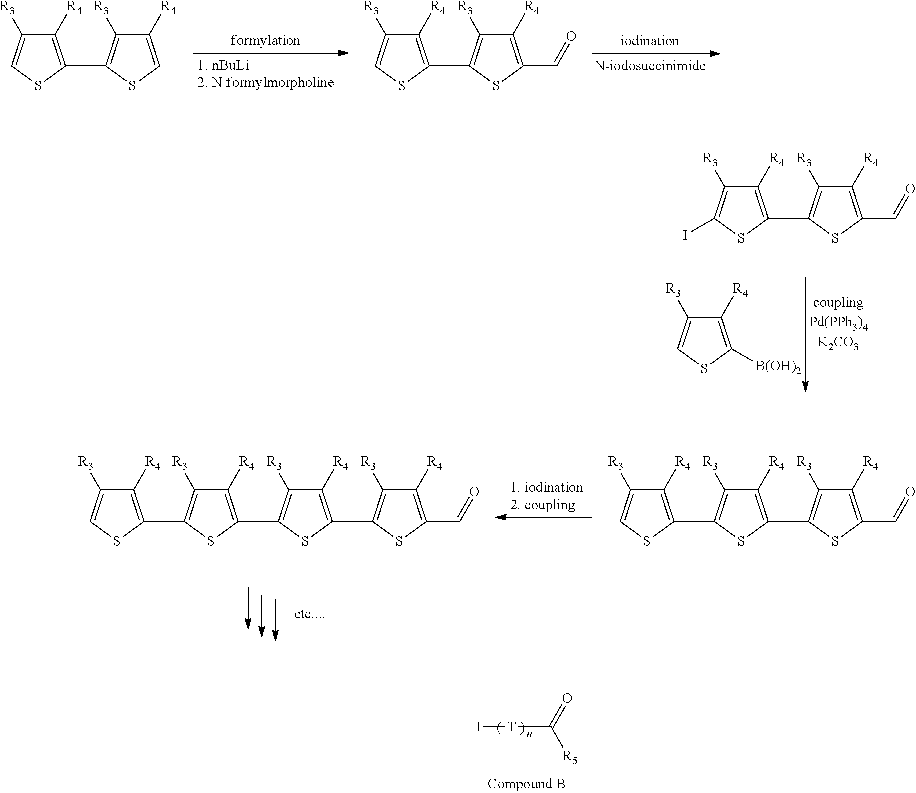

First a formylation is performed and then a cycle of iodination and Suzuki coupling is undertaken. It is understood that simple variation such as the use of a dithiophene boronic acid/ester or tristhiophene boronic acid/ester would allow oligothiophene length increases of two and three thiopehene units respectively in each cycle. It is also understood that simple variation such as the use of a fused dithiophene boronic acid/ester or a fused tristhiophene boronic acid/ester would allow synthesis of compounds of type B comprising thiophene, fused dithiophene, fused tristhiophene or mixtures thereof. The carbonyl oligothiophene may be terminated with iodide to afford a compound of type B.

It is understood that a variety of synthetic pathways can be used to make compounds of type C and that compounds of type C may have 1 to 10 T units.

A broad range of T units with R₃ and R₄ being H can be purchased from Sigma Aldrich, Apollo Chemicals, and others, which can be conveniently converted into the starting materials such as Compound B using simpler reactions, examples of which are presented below.

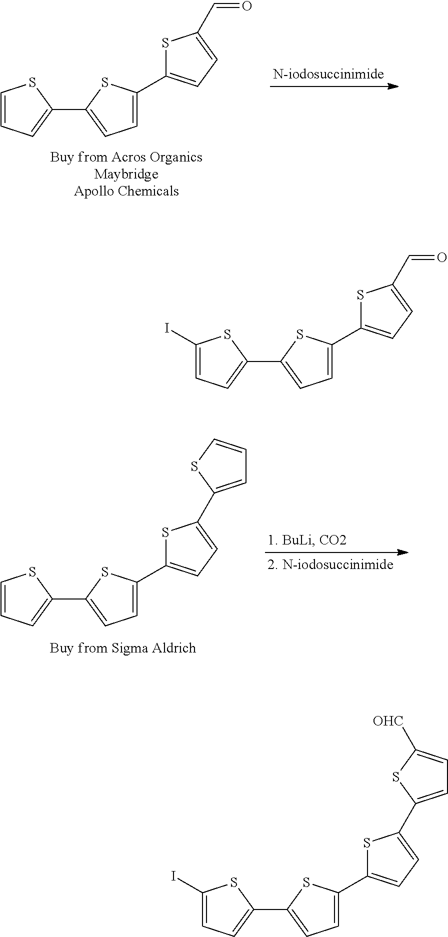 [SMILES: O=Cc1ccc(-c2ccc(-c3cccs3)s2)s1 ; O=Cc1ccc(-c2ccc(-c3ccc(I)s3)s2)s1 ; c1csc(-c2ccc(-c3ccc(-c4cccs4)s3)s2)c1 ; O=Cc1ccc(-c2ccc(-c3ccc(-c4ccc(I)s4)s3)s2)s1]

Compounds of type C can also be prepared according to any one of the following schemes:

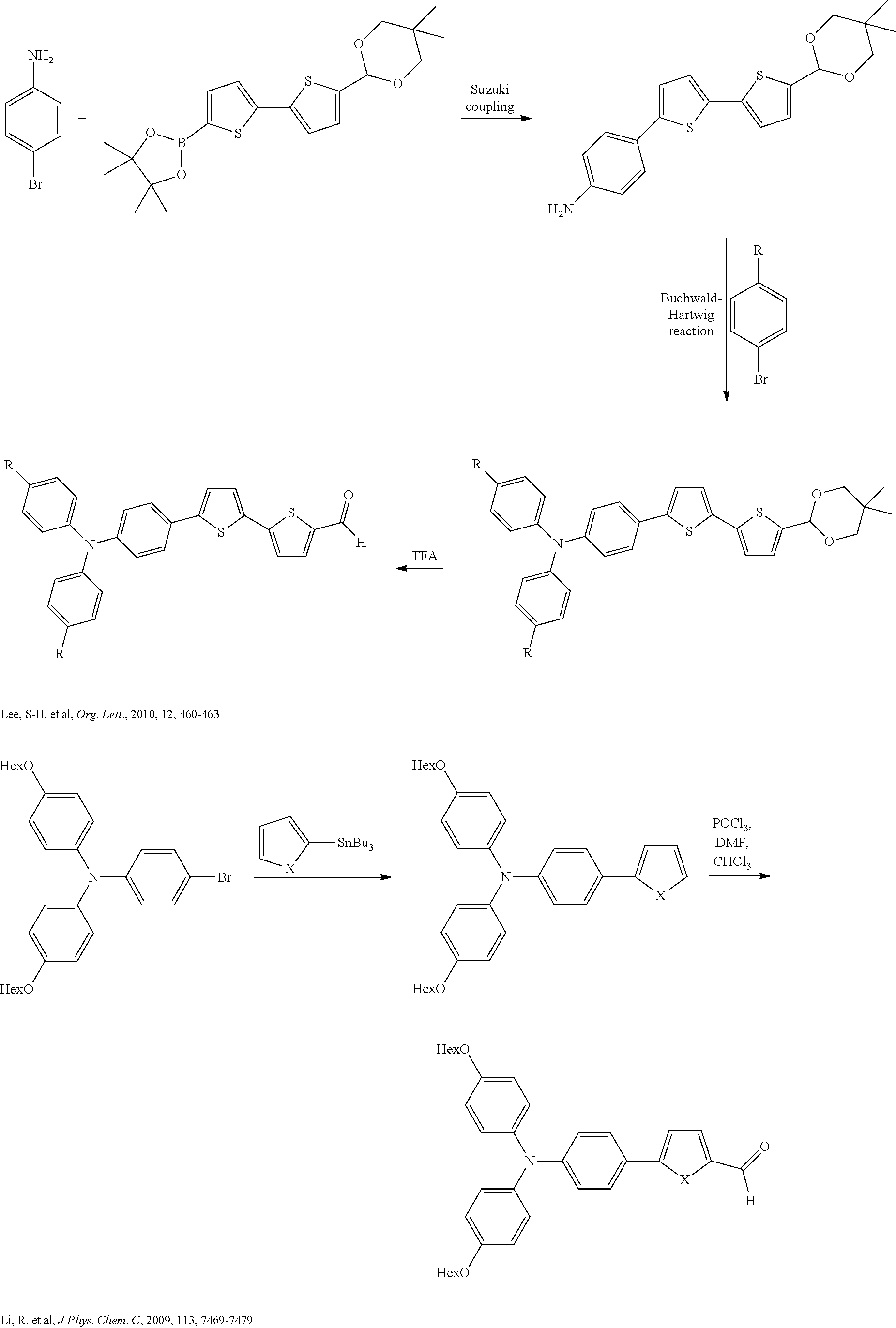

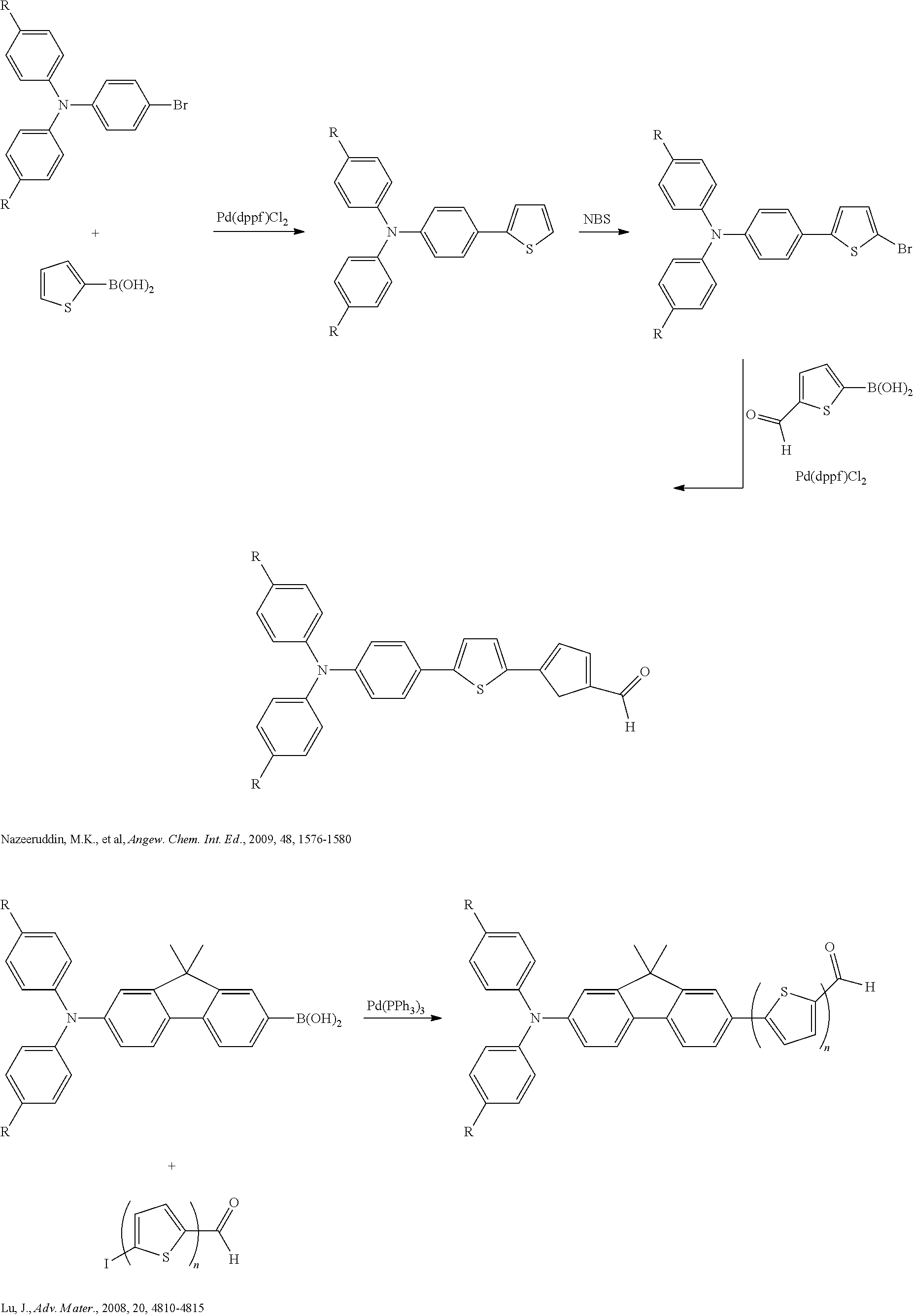

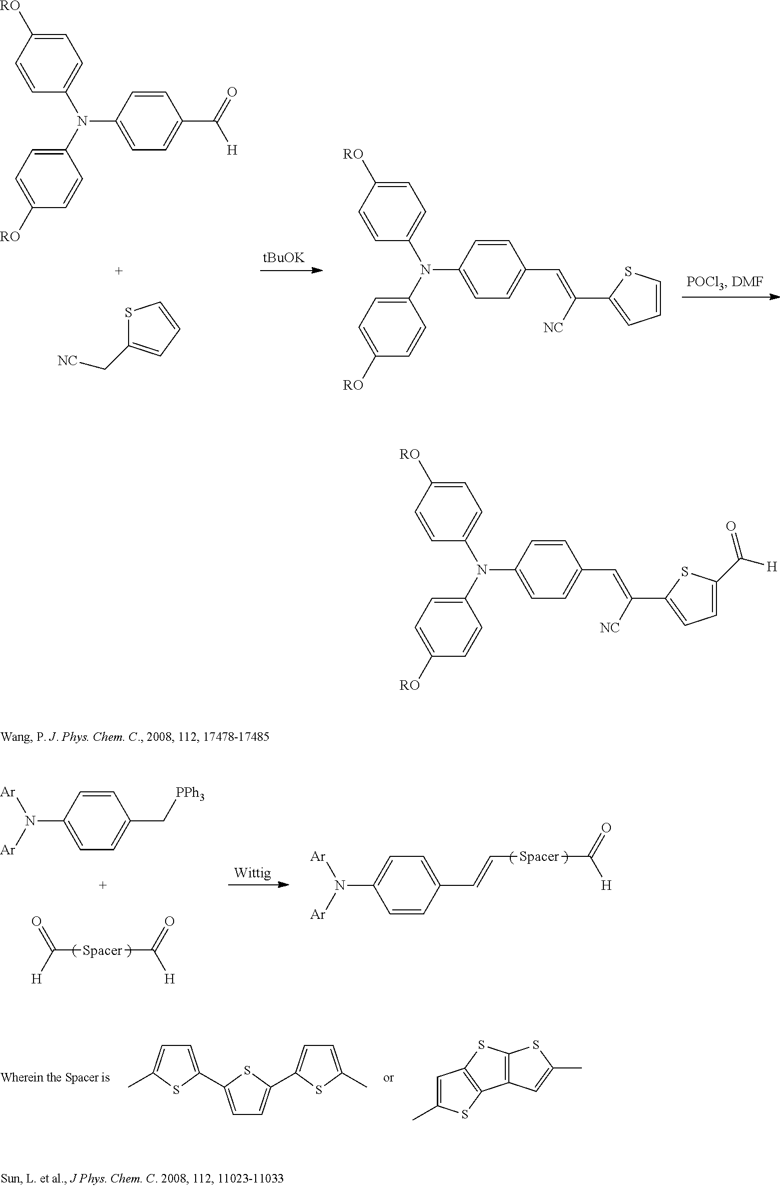

Compounds of type D can be prepared by techniques known in the art from a corresponding amine with appropriate R₇ substituents. This is demonstrated by the following scheme:

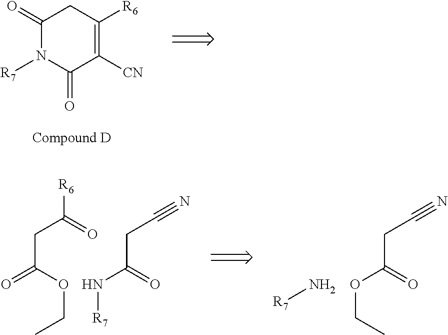

The carbonyl precursor (Compound C) can then be reacted with the cyanopyridone (Compound D) to form the target compound of formula I.

Examples of suitably substituted amine starting materials include

aminohexanol, glycine, aminobenzoic acid, allyl amine, aminotrimethylsilane, 3-aminopropylpentamethyldisiloxane, 2-ethylhexylamine, dopamine, amino acids, tris(hydroxymethyl)aminomethane (TRIS), 2-methoxyethylamine, 2-(2-aminoethoxy)ethanol and propargylamine.

Cyanopyridones have an active methylene group with acidic hydrogen atoms which react readily with aldehydes and ketones as shown below. This is frequently as simple as refluxing Compound C in an alcohol with Compound D however, a catalyst (amine base such as piperidine) or dehydrating agent (such as acetic acid or acetic anhydride) may be required. A microwave reactor can also be used.

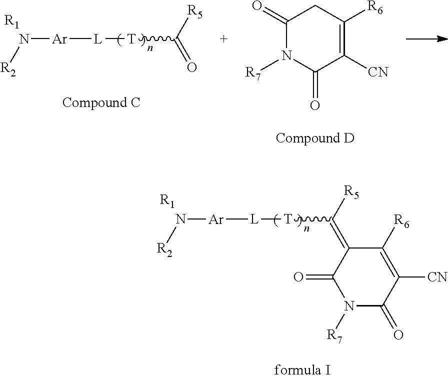

Compounds of type D or compounds of formula I which possess a reactive functional group on the R₇ substituent derived from the substituted amine starting material may have subsequent chemistry undertaken on the functional group.

### Photovoltaic Devices

The compound of formula I outlined above is suitably used in a photovoltaic device. The photovoltaic device generally comprises:
a first electrode,
a second electrode, and
an active material in electrical contact with the first and second electrodes, the active material comprising
(i) the compound of formula I, and
(ii) a second material which is a charge accepting material.

The device generates an electrical potential upon the absorption of photons. In other words, the active material is arranged such that the device generates an electrical potential upon the absorption of the photons.

The compounds of formula I may be seen as being “ambi-polar”, and may act either as an electron donor material or an electron acceptor material, depending on the relative HOMO and SUMO levels of the compound and those of the second material. In some embodiments, the compound of formula I is an electron donor and the second material is an electron acceptor. In other embodiments, the compound of formula I is an electron acceptor, and the second material is an electron donor.

The charge accepting material maybe either an electron donor material or an electron acceptor material.

Where the second material is an electron acceptor material, the material may be selected from any electron acceptor materials known in the art. The materials are generally organic electron acceptors, such as the fullerenes of various sizes (C60, C70, C80 and their soluble analogues PC61BM, PC71BM, PC84BM etc)

Where the second material is an electron donor material, the material may be selected from any electron donor materials known in the art. The materials are generally organic electron donors, such as conductive polymers including polythiophenes (including P3HT) and the like.

The photovoltaic device may be in the form of an organic solar cell, such as a bulk heterojunction organic solar cell, a bilayer organic solar cell, or a dye sensitised solar cell.

In the case of bilayer organic solar cells, the compound of formula I and the second material form layers.

In the case of a bulk heterojunction photovoltaic cell, the electron donor material (p-type conductor) and electron acceptor material (n-type conductor) are presented in a tight blend in an active material layer of the device. According to one embodiment, the concentration of each component gradually increases when approaching to the corresponding electrode.

The first electrode may be an anode. Any suitable anode materials can be used. The anode material is suitably a transparent anode material. According to some embodiments the anode is a metal oxide anode, including doped metal oxides, such as indium tin oxide, doped tin oxide, doped zinc oxide (such as aluminium-doped zinc oxide), metals such as gold, alloys and conductive polymers and the like. The anode may be supported on a suitable support. Supports include transparent supports, such as glass or polymer plates.

The second electrode may be a cathode. Any suitable cathode material can be used. According to some embodiments the cathode is a metal or metal alloy. Suitable metals and alloys are well known in the art and include aluminium, lithium, and alloys of one or both.

The device may further comprise any additional features known in the art. Some photovoltaic devices contain interfacial layers between one or both of the anodes and the active material, and such features may be incorporated in to the photovoltaic devices of the present application. The devices may be constructed by any techniques known in the art.

In the context of dye sensitised solar cells, the compound of formula I is a sensitiser, and the second material is an inorganic semiconductor material. Suitable n-type inorganic semiconductor materials are well known in the art, and include titanium dioxide (TiO₂). Suitable p-type inorganic semiconductor materials are well known in the art and include nickel oxide. The second material is suitably a particulate material. The particulate second material provides a high surface area for the attachment of molecules of the compound of formula I, which allows for high exposure to the incident light, and to high contact between the molecules of formula I and the electrolyte. Particles of a nanometer size are particularly suited, and encompass particles of between 0.1 nm to 100 nm in size, such as between 1 and 50 nm sized particles.

When the device is a dye sensitised solar cell according to some embodiments, the photovoltaic device comprises a charge transport material, which may be solid or liquid, such as an electrolyte, in contact with the compound of formula I and the second electrode. Suitable electrolytes are well known in the art and include room temperature ionic liquids, organic electrolytes and aqueous electrolytes. The electrolytes may be doped with a charge carrying species. Suitable electrolytes include iodide electrolytes.

According to some embodiments, the photovoltaic device is a dye sensitised solar cell, comprising:
an anode
a cathode,
a charge accepting material on one electrode,
a compound of formula I in contact with the charge accepting material, and
a charge transport material in contact with the compound of formula I and the other electrode.

The preferred features of the dye sensitised solar cell are as described previously in the context of photovoltaic devices. In such devices, the compound of formula I acts as a “sensitiser”.

In another embodiment the photovoltaic device is in the form of a photodetector. The photodetector comprises two electrodes and the compound of formula I (and thus has a similar structure to solar cells), and produces variations in current or voltage output in response to light.

### EXAMPLES

The present invention will now be described in further detail with reference to the following examples, relating to some embodiments of the invention. It will be understood that the invention is not limited to the embodiments provided by way of example.

### Compound Example 1

### Step 1. Synthesis of Compound 1′

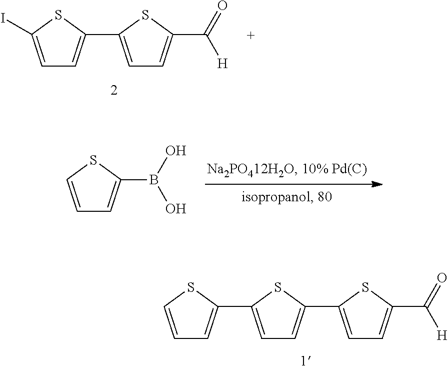 [SMILES: O=Cc1ccc(-c2ccc(I)s2)s1 ; OB(O)c1cccs1 ; O=Cc1ccc(-c2ccc(-c3cccs3)s2)s1]

5′-Iodo-2,2′-bisthiophene-5-carbaldehyde (7.0 g, 21.86 mmol), thiophene boronic acid (5.6 g, 43.72 mmol), sodium phosphate dodecahydrate (10.0 g, 21.86 mmol) and 10% Pd(C), 1.0 g, were mixed at room temperature. To this mixture was added isopropanol (150 ml) and the mixture was heated at 80° C. in an oil bath for 4 Hrs. The reaction progress was followed by TLC analysis which indicated consumption of the 5′-Iodo-2,2′-bisthiophene-5-carbaldehyde starting material. The mixture was cooled to room temperature and filtered through celite (3.0 g) eluted with dichloromethane. The solvent was removed and the residue purified by flash chromatography eluted with 50% CH₂Cl₂/petroleum ether to 100% CH₂Cl₂ to afford the title product as an orange solid, 5.60 g (20.25 mmol) 93% yield.

¹H NMR (400 MHz, CD₂Cl₂) δ 8.67 (s, 1H), 7.75 (d, 1H J=9.50 Hz), 7.33-7.30 (m, 2H), 7.27 (d, 1H J=4.00 Hz), 7.28-7.26 (dd, 1H, J₁=1.12. Hz, J₂=4.76 Hz), 7.18 (d, 1H, J=4.00 Hz), 7.09-7.60 (dd, 1H, J₁=3.60. Hz, J₂=8.76 Hz).

### Step 2. Synthesis of 5″-Iodo-[2,2′;5′,2″]terthiophene-5-carbaldehyde

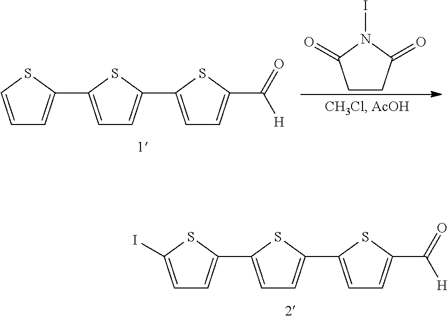

Compound 1′ (5.8 g, 20.97 mmol) was dissolved in mixture of chloroform and acetic acid 3:1 ratio (180 ml) with the aid of gentle heating and N-iodosuccinimide (5.6 g, 25.16 mmol) was added portion wise. The reaction mixture was vigorously stirred overnight and the progress of reaction was followed by TLC. The orange solid product was collected by filtration, washed with ether (3×30 ml) and dried in vacuum pump. Yield (7.4 g, 88%).

¹H NMR (200 MHz, CD₂Cl₂) δ 9.87 (s, 1H), 7.98 (d, 1H J=4.0 Hz), 7.54 (t, 2H, H J=3.3 Hz), 7.33 (d, 2H J=3.7 Hz), 7.13 (d, 1H, J=3.7 Hz).

### Step 3. Synthesis of Compound 3′

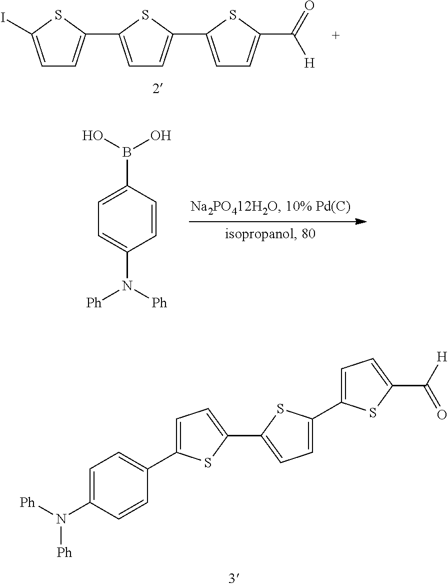 [SMILES: O=Cc1ccc(-c2ccc(-c3ccc(I)s3)s2)s1 ; OB(O)c1ccc(N(c2ccccc2)c2ccccc2)cc1 ; O=Cc1ccc(-c2ccc(-c3ccc(-c4ccc(N(c5ccccc5)c5ccccc5)cc4)s3)s2)s1]

5″-Iodo-[2,2′;5′,2″]terthiophene-5-carbaldehyde (2.5 g, 6.2 mmol), 4-(diphenylamino)phenylboronic acid (2.5 g, 8.7 mmol), sodium phosphate dodecahydrate (2.6 g, 6.8 mmol) and 10% Pd(C) (0.200 g) were mixed at room temperature. To this mixture was added isopropanol (600 ml) and the mixture was heated at 80° C. in oil bath for 24 Hrs. the reaction progress was followed by TLC analysis which indicated the consumption of 5″-iodo-[2,2′;5′,2″]-terthiophene-5-carbaldehyde. The mixture was cooled to room temperature and filtered through celite (2.0 g) eluted with dichloromethane. The solvent was removed and the residue was purified by flash chromatography eluted with 50% CHCl₃/petroleum ether to 100% CHCl₃ to afford the title product as an orange solid, 2.40 g (4.6 mmol) 74% yield.

¹H NMR (400 MHz, CD₂Cl₂) δ 9.86 (s, 1H), 7.70 (d, 1H J=13.8 Hz), 7.55-7.48 (m, 2H), 7.36-7.28 (m, 6H), 7.24-7.17 (m, 3H), 7.14-7.05 (m, 8H,).

### Step 4. Synthesis of 1-(2-ethylhexyl)-4-methyl-2,6-dioxo-1,2,5,6-tetrahydropyridine-3-carbonitrile

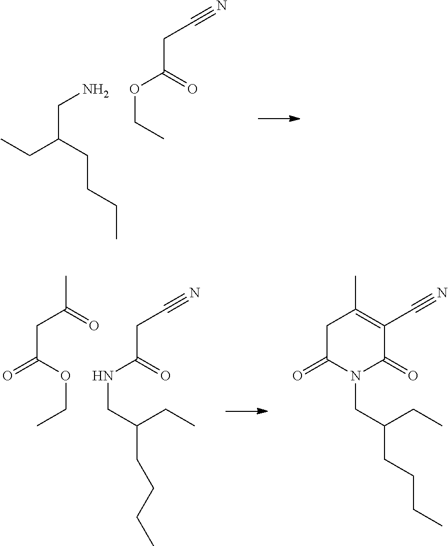

2-ethylhexyl amine (38.7 g, 0.3 moles) was added to a 500 mL round bottom flask and cooled to 0° C. Ethyl cyanoacetate (28.3 g, 0.25 moles) was added drop wise such that the solution remained cool. The reaction was stirred at room temperature overnight. Next day a mixture of ethyl acetoacetate (39.55, 0.25 moles) and piperidine (25 ml, 0.25 moles) were added drop wise and the reaction was heated at 100° C. overnight. Next day the reaction was cooled and the diluted with water before acidifying with conc. hydrochloric acid. The thick precipitate was collected by filtration and was very thick and sticky. The precipitate was dried in air as much as possible before being recrystallised from ethyl acetate and ethanol to give 32 grams of 1-(2-ethylhexyl)-4-methyl-2,6-dioxo-1,2,5,6-tetrahydropyridine-3-carbonitrile as pale pink solid.

¹H NMR (200 MHz, DMSO) δ 0.7 (m, methyl), 1.2 (m, CH₂), 2.20 (s, methyl), 3.85 (m, CH2-N), 5.6 (s, CH) (compound exists as an enol in DMSO).

### Step 5. Synthesis of Compound Example 1—(Compound 5′)

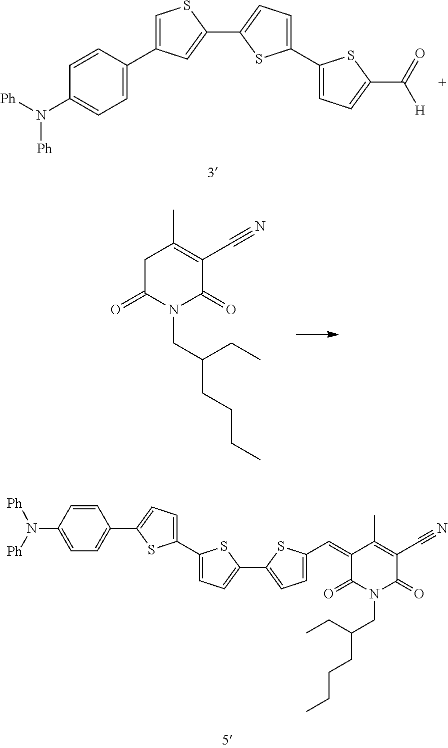 [SMILES: O=Cc1ccc(-c2ccc(-c3cc(-c4ccc(N(c5ccccc5)c5ccccc5)cc4)cs3)s2)s1 ; CCCCC(CC)CN1C(=O)CC(C)=C(C#N)C1=O ; CCCCC(CC)CN1C(=O)C(C#N)=C(C)/C(=C/c2ccc(-c3ccc(-c4ccc(-c5ccc(N(c6ccccc6)c6ccccc6)cc5)s4)s3)s2)C1=O]

Compound 3′ (0.420 g, 0.81 mmol) and 1-(2-ethylhexyl)-4-methyl-2,6-dioxo-1,2,5,6-tetrahydropyridine-3-carbonitrile (0.340 g, 1.3 mmol) were placed in a microwave reactor tube (10.0-20.0 ml) and to this mixture dichloromethane (12 ml) was added followed by pyridine (0.160 g). The mixture was heated at 75° C. for two hours in a microwave reactor (Initiator™ Biotage microwave reactor). The progress of reaction was followed by TLC which indicated that all of compound 3′ had been consumed. The dark blue reaction mixture was cooled, methanol (20 ml) was added and the product was collected by filtration and washed with hot methanol (3×30 ml). The crude material was purified by flash chromatography eluted with 50% DCM/pet. ether to 100% DCM.

Yield 250 mg and was 96% pure by HPLC.

¹H NMR (400 MHz, CD₂Cl₂) δ 7.93 (s, 1H), 7.75 (d, 1H J=4.4 Hz), 7.52-7.42 (m, 3H), 7.33-7.27 (m, 6H), 7.23 (dd, 1H, J₁=4.00. Hz, J₂=10.76 Hz), 7.14-7.06 (m, 9H), 3.97-3.93 (m, 2H), 2.63 (s, 3H), 1.89-1.84 (m, 1H), 1.36-1.32 (m, 8H), 0.95-0.91 (m, 6H).

### Compound Example 2

### Step 1. Synthesis of 5′-Iodo-2,2′-bithiophene-5-carbaldehyde

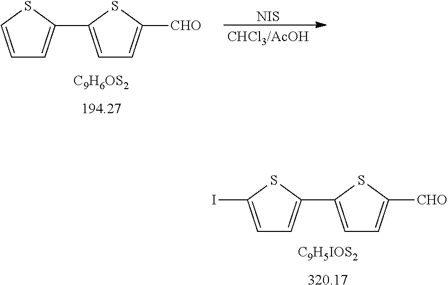

2,2′-bithiophene-5-carbaldehyde (2.0 g, 10.31 mmol) was added to a stirred 1:1 (v/v) solvent mixture of chloroform and acetic acid (30 ml) in a 100 ml RB flask at room temperature followed by the addition of N-iodosuccinimide (1.2 Eq, 12.37 mmol, 2.8 g). The resulting reaction mixture was stirred at room temperature overnight. A solid appeared in the reaction which was filtered off and washed with pre-cooled acetic acid followed by diethyl ether, to give the titled compound (2.9 g, 87.91%) as yellow powder.

¹H NMR: (400 MHz, DMSO) δ 9.93 (s, 1H), δ 7.91 (d, 1H, J=4 Hz), δ 7.44 (m, 1H), δ 7.38 (m, 1H), δ 7.24 (m, 1H).

### Step 2. Synthesis of 5′-(4-(diphenylamino)phenyl)-2,2′-bithiophene-5-carbaldehyde

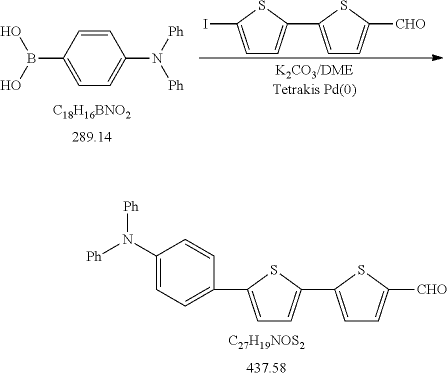

5′-Iodo-2,2′-bithiophene-5-carbaldehyde (2.5 g, 7.81 mmol) was added to a mixture of 4-(diphenylamino)phenylboronic acid (3.4 g, 11.72 mmol) in dimethoxy ethane (30 ml) followed by the addition of potassium carbonate (3.3 g, 23.43 mmol). The resulting suspension was stirred and degassed for 30 minutes at ambient temperature followed by the addition of tetrakis(triphenylphosphine)palladium(0) (451 mg, 0.39 mmol). The resulting mixture was refluxed for 2 Hrs and TLC analysis indicated the presence of product. The solvent was removed under reduced pressure and the resulting crude material was subjected to column chromatography to give 1.32 g (38.7%) of the titled product as an orange powder.

¹H NMR: (400 MHz, DMSO) δ 9.86 (s, 1H), δ 7.70 (m, 1H), δ 7.50 (m, 2H), δ 7.36 (m, 1H), δ 7.33-7.28 (m, 5H), δ 7.23 (m, 1H), δ 7.14-7.05 (m, 8H).

### Step 3. Synthesis of Compound Example 2, 5-((5′-(4-(diphenylamino)phenyl)-2,2′-bithiophen-5-yl)methylene)-1-(2-ethylhexyl)-4-methyl-2,6-dioxo-1,2,5,6-tetrahydropyridine-3-carbonitrile

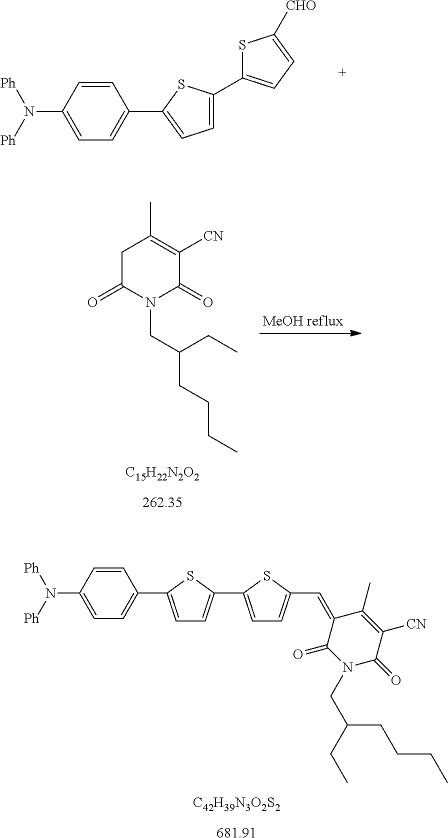

5′-(4-(diphenylamino)phenyl)-2,2′-bithiophene-5-carbaldehyde (104 mg, 0.24 mmol) and 1-(2-ethylhexyl)-4-methyl-2,6-dioxo-1,2,5,6-tetrahydropyridine-3-carbonitrile (113 mg, 0.432 mmol) were taken in methanol (20 ml) in a round bottom flask at ambient temperature and the resulting mixture was heated at reflux overnight. A solid appeared in the reaction which was filtered off and washed with methanol to afford 125 mg, 77.3% of the Compound Example 2 as a black powder.

¹H NMR: (400 MHz, DMSO) δ 8.33 (s, 1H), δ 8.21 (d, J=4.4 Hz, 1H), δ 7.71-7.74 (m, 2H), δ 7.65 (m, 2H), δ 7.52 (m, 1H), δ 7.36-7.323 (m, 4H), δ 7.12-7.06 (m, 6H), δ 6.95 (m, 2H), δ 3.79 (m, 2H), δ 2.6 (s, 3H), δ 1.80-1.70 m, 1H), δ 1.35-1.15 (m, 8H), δ 0.90-0.81 (m, 6H); found m/z=681.2; UV-Vis (CH₂Cl₂ film) λ max 584 nm (Onset 790 nm).

### Compound Example 3

### Step 1. Synthesis of 4-(2-ethylhexyl)-4H-dithieno[3,2-b:2′,3′-d]pyrrole

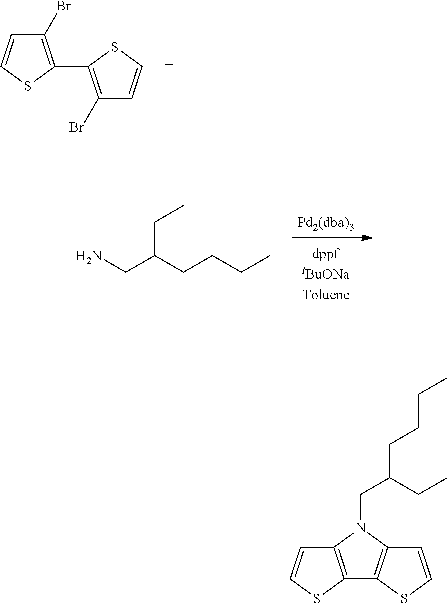

3,3′-dibromo-2,2′-bithiophene (715 mg, 2.22 mmol) was taken in toluene (25 ml) in a 100 ml round bottom flask followed by the addition of sodium-t-butoxide (508 mg, 5.29 mmol) at room temperature (RT). Pd catalyst (101.6 mg, 0.11 mmol) was added to this mixture followed by the addition of ligand dppf (244 mg, 0.44 mmol) at RT and the resulting reaction mixture was stirred for 15 minutes followed by the addition of 2-ethylhexylamine (286 mg, 2.22 mmol) at RT. The resulting reaction mixture was heated to reflux for overnight and solvent was evaporated under reduced pressure to obtain crude yellow oil which was subjected to column chromatography on silica (hexane:dichloromethane (9:1) to afford 540 mg (83.9%) of the product as colorless oil.

¹H NMR (CDCl₃, 400 MHz): δ 7.13 (m, 2H), 6.99 (m, 2H), 4.11-4.01 (m, 2H), 1.99-1.92 (m, 1H), 1.40-1.23 (m, 8H), 0.92-0.86 (m, 6H)

### Step 2. Synthesis of 4-(2-ethylhexyl)-4H-dithieno[3,2-b:2′,3′-d]pyrrole-2-carbaldehyde

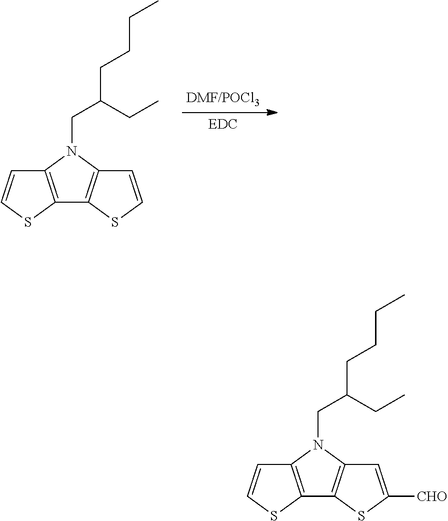 [SMILES: CCCCC(CC)Cn1c2ccsc2c2sccc21 ; CCCCC(CC)Cn1c2ccsc2c2sc(C=O)cc21]

4-(2-ethylhexyl)-4H-dithieno[3,2-b:2′,3′-d]pyrrole (540 mg, 1.86 mmol) from step 1 was taken in ethylene dichloride (25 ml) in a 100 ml round bottom flask followed by the addition of dimethylformamide (149.3 mg, 2.05 mmol) at RT. The resulting reaction solution was cooled to 0° C. and POCl₃ (0.51 ml, 5.58 mmol) was added to it. The reaction mixture was allowed to warm to RT and refluxed for overnight. The reaction mix was allowed to cool down, worked up with saturated sodium acetate solution and the product extracted in CHCl₃. The organic layer was washed twice with water followed by brine and dried over Na₂SO₄ and recovered to get crude yellow oil which was subjected to column chromatography on silica (Hexane:Ethyl acetate (9:1)) to afford 540 mg (75.8%) of desired compound 4-(2-ethylhexyl)-4H-dithieno[3,2-b:2′,3′-d]pyrrole-2-carbaldehyde as dark yellow oil. ¹H NMR (CDCl₃, 200 MHz): δ 9.88 (s, 1H), 7.63 (s, 1H), 7.36 (m, 1H), 6.98 (m, 1H), 4.11-4.08 (m, 2H), 2.04-1.88 (m, 1H), 1.41-1.21 (m, 8H), 0.95-0.81 (m, 6H)

### Step 3. Synthesis of 4-(2-ethylhexyl)-6-iodo-4H-dithieno[3,2-b:2′,3′-d]pyrrole-2-carbaldehyde

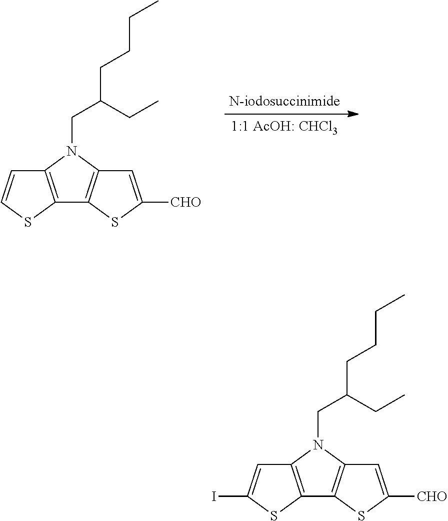 [SMILES: CCCCC(CC)Cn1c2ccsc2c2sc(C=O)cc21 ; CCCCC(CC)Cn1c2cc(I)sc2c2sc(C=O)cc21]

### 4-(2-Ethylhexyl)-4H-dithieno[3,2-b:2′,3′-d]pyrrole-2-carbaldehyde

(450 mg, 1.41 mmol) from step 2 was taken in 1:1 solvent mixture (25 ml) of acetic acid and chloroform in a 100 ml round bottom flask followed by the addition of N-iodosuccinimide (412 mg, 1.83 mmol) at RT. The resulting reaction solution was stirred in the dark at RT for overnight. The reaction mixture was worked up with water and chloroform and the organic layer was separated, washed with 20% sodium thiosulphate followed by water and brine, dried over anhydrous Na₂SO₄ and recovered to afford the product 4-(2-ethylhexyl)-6-iodo-4H-dithieno[3,2-b:2′,3′-d]pyrrole-2-carbaldehyde as crude dark brown oil (400 mg, 63.7%) which solidified at RT. ¹H NMR (CDCl₃, 200 MHz): δ 9.88 (s, 1H), 7.60 (s, 1H), 7.19 (s, 1H), 4.08-4.02 (m, 2H), 1.99-1.84 (m, 1H), 1.40-1.17 (m, 8H), 0.95-0.84 (m, 6H).

### Step 4. Synthesis of 64-(diphenylamino)phenyl-4-(2-ethylhexy)-4H-dithieno[3.2-b:2′,3′-d]pyrrol-2-carbaldehyde

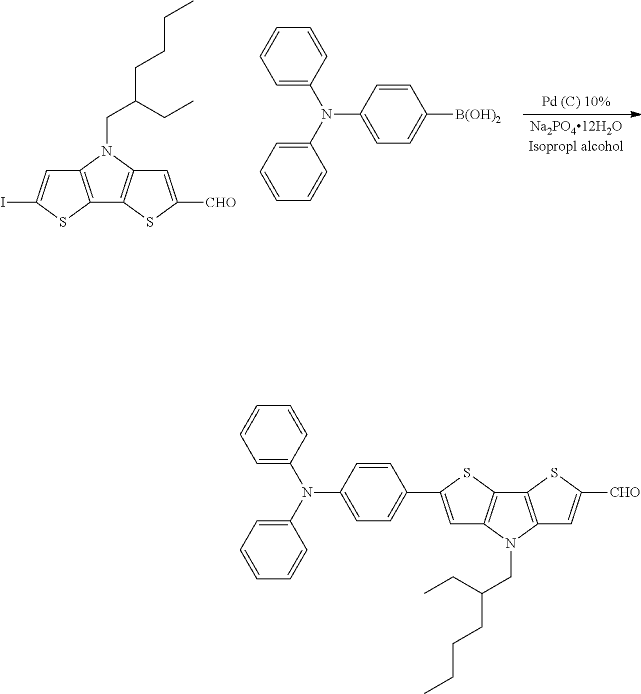 [SMILES: CCCCC(CC)Cn1c2cc(I)sc2c2sc(C=O)cc21 ; OB(O)c1ccc(N(c2ccccc2)c2ccccc2)cc1 ; CCCCC(CC)Cn1c2cc(C=O)sc2c2sc(-c3ccc(N(c4ccccc4)c4ccccc4)cc3)cc21]

4-(2-ethylhexyl)-6-iodo-4H-dithieno[3,2-b:2′,3′-d]pyrrole-2-carbaldehyde (843 mg, 1.89 mmol) from step 3,4-(diphenylamino)phenylboronic acid (927 mg, 3.21 mmol), sodium phosphate dodecahydrate (1686 mg, 4.72 mmol) and 10% Pd(C) (640 mg) were mixed in isopropanol (100 ml) in 250 ml RB flask at RT. The mixture was heated to 80° C. in oil bath for 24 Hrs and the reaction progress was followed by thin-layer chromatography (TLC), which indicated the consumption of starting aldehyde. The reaction mixture was filtered off and the solvent was recovered to get crude oil which was subjected to column chromatography on silica (Hexane:Ethyl acetate (8:2)) to afford 700 mg (66.1%) of 6-(4-(diphenylamino)phenyl-4-(2-ethylhexyl)-4H-dithieno[3,2-b:2′,3′-d]pyrrol-2-carbaldehyde as orange oil. ¹H NMR (CD₂Cl₂, 200 MHz): δ 9.86 (s, 1H), 7.65 (s, 1H), 7.58 (m, 2H), 7.35-7.28 (m, 4H), 7.19-7.02 (m, 9H), 4.15-4.11 (m, 2H), 2.06-1.96 (m, 1H), 1.38-1.21 (m, 8H), 0.97-0.87 (m, 6H).

### Step 5. Synthesis of 5-((6-(4-(diphenylamino)phenyl)-4-(2-ethylhexyl)-4H-dithieno[3,2-b:2′,3′-d]pyrrol-2-yl)methylene)-1-(2-ethylhexyl)-4-methyl-2,6-dioxo-1,2,5,6-tetrahydropyridine-3-carbonitrile

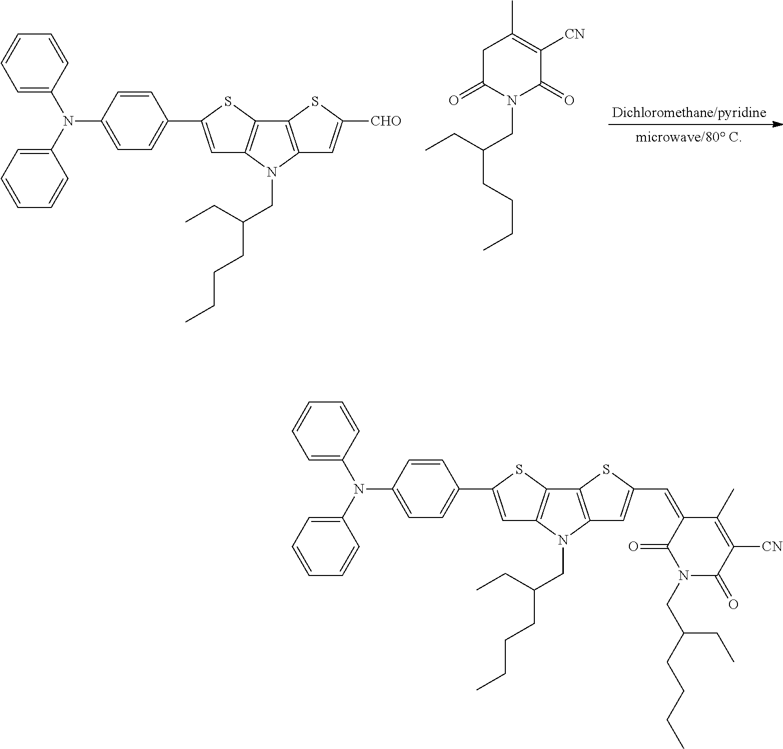 [SMILES: CCCCC(CC)Cn1c2cc(C=O)sc2c2sc(-c3ccc(N(c4ccccc4)c4ccccc4)cc3)cc21 ; CCCCC(CC)CN1C(=O)CC(C)=C(C#N)C1=O ; CCCCC(CC)CN1C(=O)C(C#N)=C(C)/C(=C/c2cc3c(s2)c2sc(-c4ccc(N(c5ccccc5)c5ccccc5)cc4)cc2n3CC(CC)CCCC)C1=O]

6-(4-(diphenylamino)phenyl-4-(2-ethylhexyl)-4H-dithieno[3,2-b:2′,3′-d]pyrrol-2-carbaldehyde (700 mg, 1.24 mmol) from step 4 and 1-(2-ethylhexyl)-4-methyl-2,6-dioxo-1,2,5,6-tetrahydropyridine-3-carbonitrile (550 mg, 2.2 mmol) were placed in a microwave reactor tube (10.0-20.0 ml) and dichloromethane (15 ml) was added to this mixture followed by the addition of pyridine (147 mg, 1.86 mmol). The mixture was heated at 80° C. for two hours in a microwave reactor (Initiator™ Biotage microwave reactor). The progress of reaction was followed by TLC, which indicated the complete consumption of 6-(4-(diphenylamino)phenyl-4-(2-ethylhexyl)-4H-dithieno[3,2-b:2′,3′-d]pyrrol-2-carbaldehyde. The dark blue reaction mixture was cooled and the solvent was evaporated off. Ethanol (20 ml) was added and the product was collected by filtration and washed with hot ethanol (3×30 ml). The crude material was purified by flash chromatograph eluted with dichloromethane (100% DCM→2% Ether/DCM) to afford 550 mg (54.8%) of titled compound as dark bluish-black shiny powder.

HPLC (5% THF/ACN): 96%; M. Pt.: 137-140° C.; IR (neat, cm⁻¹) 2950, 2924, 2856, 2217, 1678, 1630, 1589, 1570, 1486, 1447, 1413, 1291, 1282, 1171, 783, 751, 694; ¹H NMR (400 MHz, CD₂Cl₂) δ 7.95 (s, 1H), 7.79 (broad s, 1H), 7.54-7.52 (m, 2H), 7.33-7.27 (m, 4H), 7.15-7.10 (m, 7H), 7.06-7.04 (m, 2H), 4.09-4.06 (m, 2H), 3.98-3.80 (m, 2H), 2.59 (s, 3H), 2.01-1.95 (m, 1H), 1.89-1.83 (m, 1H), 1.40-1.24 (m, 16H) 0.95-0.81 (m, 12H).

¹³NMR (200 MHz, CDCl₃): δ 163.24, 161.46, 157.88, 153.82, 152.50, 148.78, 146.95, 146.09, 144.52, 136.09, 134.46, 129.46, 127.48, 126.75, 125.64, 125.10, 123.83, 122.47, 115.87, 114.30, 113.69, 105.07, 100.60, 51.41, 43.99, 40.26, 37.42, 30.62, 30.56, 28.60, 28.55, 24.06, 23.87, 23.09, 22.94, 18.97, 14.08, 13.98, 10.68, 10.62; HRMS/EI: calcd for C₅₀H₅₄N₄O₂S₂ (m/z) 806.3683; found=806.3646. UV-Vis (CHCl₃ solution) λ max 623 nm (onset 725 nm)

Molar extinction coefficient (ε)=˜99000 M⁻¹ cm⁻¹ UV-Vis (CHCl₃ film) λ max 611 nm (onset 796 nm). Energy gap (ΔE)=1.56 eV HOMO (PESA): −5.42 eV; LUMO: −3.86 eV

### Compound Example 4

### Step 1. Synthesis of thieno[3,2-b]thiophene-2-carbaldehyde

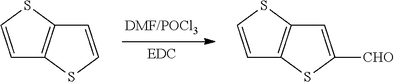 [SMILES: c1cc2sccc2s1 ; O=Cc1cc2sccc2s1]

Thieno[3,2-b]thiophene (3 g, 21.43 mmol) was taken in ethylene dichloride (75 ml) in a 250 ml round bottom flask followed by the addition of dimethylformamide (1.65 ml, 21.43 mmol) at RT. The resulting reaction solution was cooled to 0° C. and POCl₃ (5.87 ml, 64.29 mmol) was added to it. The reaction mixture was allowed to warm to RT and refluxed for overnight. Reaction mix was allowed to cool down, worked up with saturated sodium acetate solution and product was extracted in ethyl acetate. The organic layer was washed twice with water followed by brine and dried over Na₂SO₄ and recovered to afford crude yellow oil which was subjected to column chromatography on silica (Hexane:Ethyl acetate (9:1)) to afford 2.7 g (74.98%) of thieno[3,2-b]thiophene-2-carbaldehyde as light yellow oil.

¹H NMR (200 MHz, DMSO) δ 9.97 (s, 1H), 8.39 (m, 1H), 8.07 (m, 1H), 7.55 (m, 1H)

### Step 2. Synthesis of 5-iodothieno[3,2-b]thiophene-2-carbaldehyde

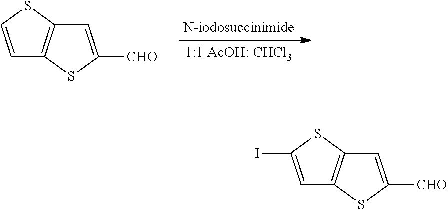 [SMILES: O=Cc1cc2sccc2s1 ; O=Cc1cc2sc(I)cc2s1]

Thieno[3,2-b]thiophene-2-carbaldehyde (1.5 g, 8.93 mmol) from step 1 was taken in 1:1 solvent mixture (50 ml) of acetic acid and chloroform in a 250 ml round bottom flask followed by the addition of n-iodosuccinimide (2.51 g, 11.16 mmol) at RT. The resulting reaction solution was stirred in the dark at RT for overnight. The solid appeared in the reaction was filtered off, washed with water followed by hexane and dried UN to afford 1 g (63.7%) of 5-iodothieno[3,2-b]thiophene-2-carbaldehyde as light green solid.

¹H NMR (200 MHz, CD₃COCD₃) δ 10.05 (s, 1H), 8.25 (s, 1H), 7.82 (s, 1H)

### Step 3. Synthesis of 5-(4-(diphenylamino)phenyl)thieno[3,2-b]thiophene-2-carbaldehyde

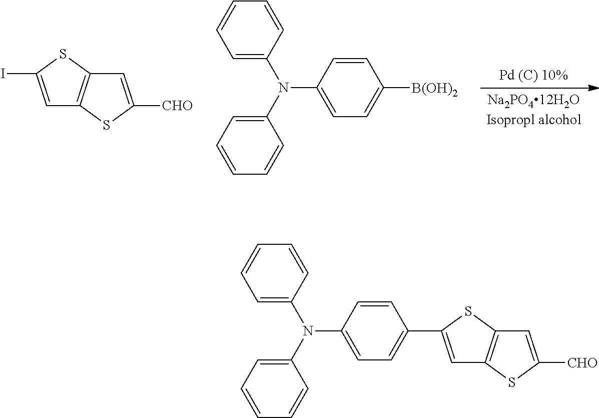 [SMILES: O=Cc1cc2sc(I)cc2s1 ; OB(O)c1ccc(N(c2ccccc2)c2ccccc2)cc1 ; O=Cc1cc2sc(-c3ccc(N(c4ccccc4)c4ccccc4)cc3)cc2s1]

5-Iodothieno[3,2-b]thiophene-2-carbaldehyde (1.0 g, 3.4 mmol)) from step 2, 4-(diphenylamino)phenylboronic acid (1.5 g, 5.1 mmol), sodium phosphate dodecahydrate (1.55 g, 4.08 mmol) and 10% Pd(C) (0.20 g) were mixed at room temperature. To this mixture isopropanol (100 ml) was added and the mixture was heated to 80° C. in oil bath for 24 Hrs and the reaction progress was followed by TLC analysis, which indicated the consumption of 5-Iodothieno[3,2-b]thiophene-2-carbaldehyde. The reaction mixture was cooled to room temperature and filtered through celite (2.0 g) eluted with dichloromethane. The solvent was removed. The crude product was purified by flash chromatograph eluted with 50% CHCl₃/petroleum ether-→CHCl₃ to give 1.1 g (78%) of 5-(4-(diphenylamino)phenyl)thieno[3,2-b]thiophene-2-carbaldehyde as yellow solid. ¹H NMR (400 MHz, CDCl₃) δ 9.93 (s, 1H), 7.88 (s, 1H), 7.51-7.48 (m, 2H), 7.42 (m, 1H), 7.31-7.27 (m, 4H), 7.15-7.13 (m, 4H), 7.10-7.6 (m, 4H)

### Step 4. Synthesis of 5-((5-(4-(diphenylamino)phenyl)thieno[3,2-b]thiophene-2-yl)methylene)-1-(2-ethylhexyl)-4-methyl-2,6-dioxo-1,2,5,6-tetrahydropyridine-3-carbonitrile

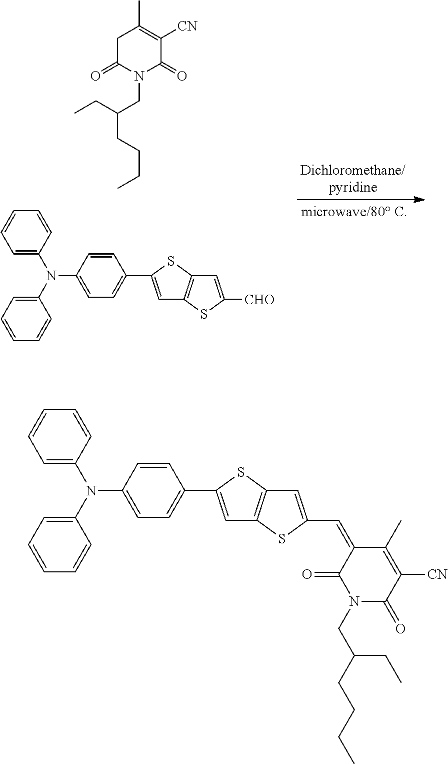 [SMILES: CCCCC(CC)CN1C(=O)CC(C)=C(C#N)C1=O ; O=Cc1cc2sc(-c3ccc(N(c4ccccc4)c4ccccc4)cc3)cc2s1 ; CCCCC(CC)CN1C(=O)C(C#N)=C(C)/C(=C/c2cc3sc(-c4ccc(N(c5ccccc5)c5ccccc5)cc4)cc3s2)C1=O]

5-(4-(diphenylamino)phenyl)thieno[3,2-b]thiophene-2-carbaldehyde (900 mg, 2.18 mmol) from step 3 and 1-(2-ethylhexyl)-4-methyl-2,6-dioxo-1,2,5,6-tetrahydropyridine-3-carbonitrile (970 mg, 3.72 mmol) were placed in a microwave reactor tube (10.0-20.0 ml) and dichloromethane (18 ml) was added to this mixture followed by the addition of pyridine (340 mg, 4.36 mmol). The mixture was heated at 80′C for two hours in a microwave reactor (Initiator™ Biotage microwave reactor). The progress of reaction was followed by TLC, which indicated the complete consumption of aldehyde. The dark blue reaction mixture was cooled and the solvent was evaporated off. Ethanol (20 ml) was added and the product was collected by filtration and washed with hot ethanol (3×30 ml). The crude material was purified by flash chromatograph eluted with 50% dichloromethane/pet ether (60-80° C.)→100% DCM→2% Ether/DCM to afford 900 mg (63%) of titled compound as greenish black shiny powder.

HPLC (5% THF/ACN): 97%; M. Pt.: 208-213° C.; IR (neat, cm⁻¹) 2950, 2924, 2856, 2217, 1678, 1630, 1589, 1570, 1486, 1447, 1413, 1291, 1282, 1171, 783, 751, 694; ¹H NMR (400 MHz, CD₂Cl₂) δ 7.96 (s, 1H), 7.91 (5, 1H), 7.54-7.51 (m, 2H), 7.44- (s, 1H), 7.33-7.29 (m, 4H), 7.16-7.05 (m, 8H), 4.01-3.90 (m, 2H), 2.64 (s, 3H), 1.89-1.85 (m, 1H), 1.37-1.26 (m, 8H), 0.93-0.85 (m, 6H); ¹³C NMR (200 MHz, CDCl₃): δ 163.15, 160.94, 157.98, 156.28, 156.12, 149.39, 146.77, 144.72, 139.19, 138.36, 136.90, 129.52, 127.28, 126.33, 125.34, 124.10, 122.02, 116.22, 115.10, 114.35, 103.27, 44.02, 37.46, 30.52, 28.48, 23.86, 23.05, 18.91, 14.07, 10.55; HRMS/EI: calcd for C₄₀H₃₇N₃C₂S₂ (m/z) 655.2322; found=655.2311. UV-Vis (CHCl₃ solution) λ max 571 nm (onset 696 nm)

Molar extinction coefficient (ε)=˜58000 M⁻¹ cm⁻¹ UV-Vis (CHCl₃ film) λ max 512 nm (onset 674 nm). Energy gap (ΔE)=1.84 eV. HOMO (PESA): −5.40 eV; LUMO: −3.56 eV

### Compound Example 5

### Step 1. Synthesis of 3,3′″-dihexyl-[2,2′:5′,2″:5″,2′″-quaterthiophene]-5-carbaldehyde

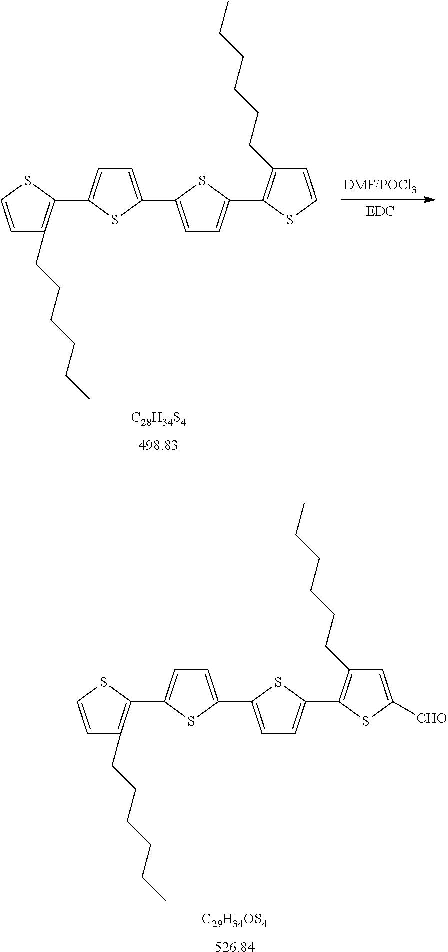 [SMILES: CCCCCCc1ccsc1-c1ccc(-c2ccc(-c3sccc3CCCCCC)s2)s1 ; "C28H34S4" ; CCCCCCc1ccsc1-c1ccc(-c2ccc(-c3sc(C=O)cc3CCCCCC)s2)s1 ; "498.83" ; "C29H34OS4" ; "526.84"]

Substrate 3,3′″-dihexyl-2,2′:5′,2″:5″,2′″-quaterthiophene (420 mg, 0.84 mmol) was taken in 100 ml RB flask in ethylene dichloride (25 ml) and dimethyl formamide (67 mg, 0.92 mmol) was added to it. The resulting reaction mix was cooled to 0° C. and POCl₃ (0.23 ml, 2.52 mmol) was added to it at this temperature. The reaction mix was allowed to warm to RT and refluxed for overnight. The reaction mix was treated with saturated sodium acetate solution and EDC layer was separated, washed with water twice followed by brine and dried over anhydrous sodium sulphate and recovered to afford crude dark yellow oil which was subjected to column chromatography on silica gel (Hexane:EtOAc (470:30 ml)) to afford 230 mg (52.02%) of deep orange oil which began to solidify at RT after some time.

¹H NMR (CDCl₃, 400 MHz): δ 9.81 (s, 1H), 7.58 (s, 1H), 7.20-7.14 (m, 4H), 7.03 (m, 1H), 6.93 (m, 1H), 2.83-2.74 (m, 4H), 1.72-1.59 (m, 4H), 1.41-1.19 (m, 12H), 0.91-0.83 (m, 6H)

### Step 2. Synthesis of 3,3′″-dihexyl-5′″-iodo-[2,2′:5′,2″:5″,2′″-quaterthiophene]-5-carbaldehyde

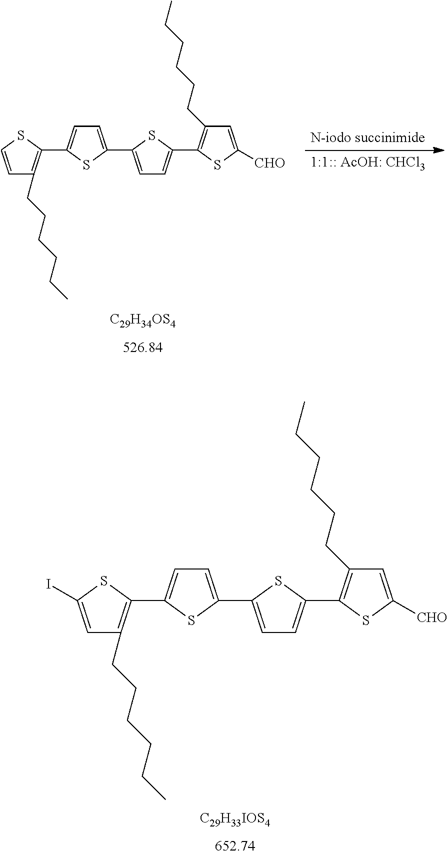 [SMILES: CCCCCCc1ccsc1-c1ccc(-c2ccc(-c3sc(C=O)cc3CCCCCC)s2)s1 ; "C29H34OS4" ; "526.84" ; CCCCCCc1cc(I)sc1-c1ccc(-c2ccc(-c3sc(C=O)cc3CCCCCC)s2)s1 ; "C29H33IOS4" ; "652.74"]

Substrate 3,3′″-dihexyl-[2,2′:5′,2″:5″,2′″-quaterthiophene]-5-carbaldehyde (230 mg, 0.44 mmol) from step 1 was taken in 100 ml RB flask in acetic acid:chloroform (1:1) (v/v) solvent mixture (20 ml) and N-iodosuccinimide was added to it at RT. The resulting reaction mix was stirred in the dark overnight at RT. The solid appeared in the reaction was filtered off and washed with hexane to get 250 mg (87.04%) of brick red solid.

¹H NMR (CDCl₃, 400 MHz): δ 9.81 (s, 1H), 7.58 (s, 1H), 7.18 (m, 1H), 7.15 (m, 2H), 7.07 (s, 1H), 6.96 (m, 1H), 2.80 (m, 2H), 2.71 (m, 2H), 1.71-1.56 (m, 4H), 1.41-1.23 (m, 12H), 0.90-0.85 (m, 6H)

### Step 3. Synthesis of 5′″-(4-(diphenylamino)phenyl)-3,3′″-dihexyl-[2,2′:5′,2″:5″,2′″-quaterthiophene]-5-carbaldehyde

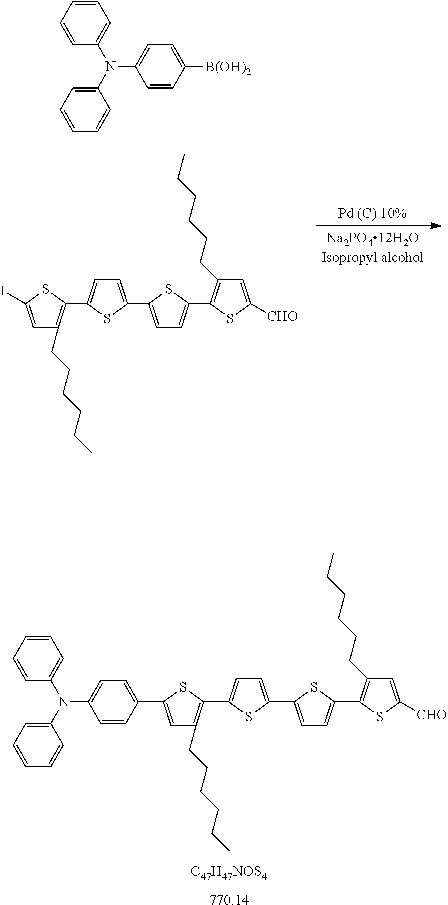 [SMILES: OB(O)c1ccc(N(c2ccccc2)c2ccccc2)cc1 ; CCCCCCc1cc(I)sc1-c1ccc(-c2ccc(-c3sc(C=O)cc3CCCCCC)s2)s1 ; CCCCCCc1cc(C=O)sc1-c1ccc(-c2ccc(-c3sc(-c4ccc(N(c5ccccc5)c5ccccc5)cc4)cc3CCCCCC)s2)s1 ; "C47H47NOS4" ; "770.14"]

3,3′″-dihexyl-5′″-iodo-[2,2′:5′,2″:5″,2′″-quaterthiophene]-5-carbaldehyde (1 g, 1.5 mmol) from step 2,4-(diphenylamino)phenylboronic acid (0.66 g, 2.3 mmol), sodium phosphate dodecahydrate (0.70 g, 1.8 mmol) and 10% Pd(C) (0.20 g) were mixed in isopropanol (100 ml) in 250 ml round bottom flask at RT. The mixture was heated to 80° C. in oil bath for 24 Hrs and the reaction progress was followed by thin-layer chromatography (TLC), which indicated the consumption of starting aldehyde.

The mixture was cooled to room temperature and filtered through celite (2.0 g) eluted with dichloromethane. The solvent was removed and the residue was purified by flash chromatography eluted with 30% CHCl₃/petroleum ether to afford 0.80 g (70%) of title product as yellow solid.

¹H NMR (CD₂Cl₂, 200 MHz): δ 9.86 (s, 1H), 7.65 (s, 1H), 7.58 (m, 2H), 7.35-7.28 (m, 4H), 7.19-7.02 (m, 9H), 4.15-4.11 (m, 2H), 2.06-1.96 (m, 1H), 1.38-1.21 (m, 8H), 0.97-0.87 (m, 6H)

### Step 4. Synthesis of 5-((5′″-(4-(diphenylamino)phenyl)-3,3′″-dihexyl-[2,2′:5′,2″:5″,2′″-quaterthiophene]-5-yl)methylene)-1-(2-ethylhexyl)-4-methyl-2,6-dioxo-1,2,5,6-tetrahydropyridine-3-carbonitrile

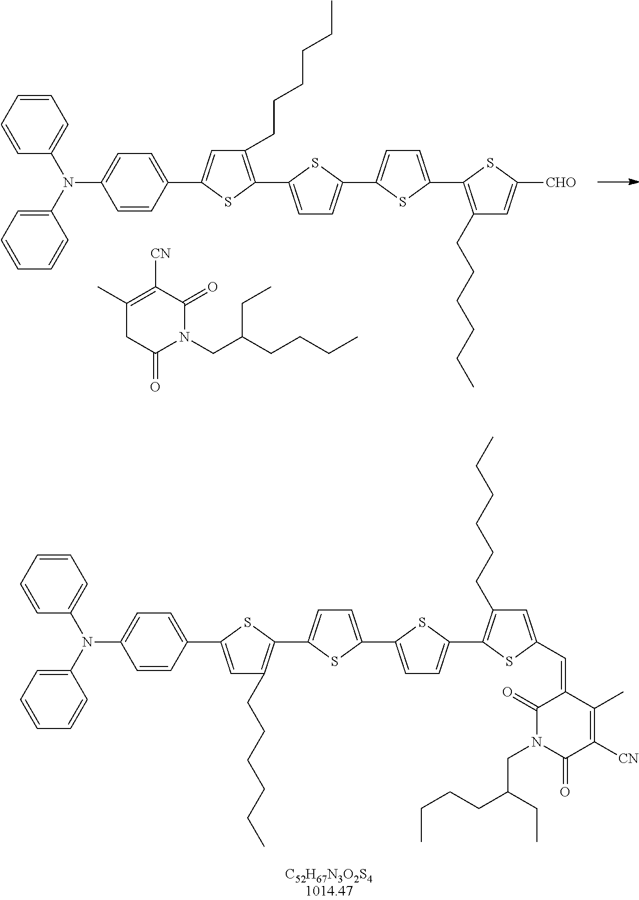

5′″-(4-(diphenylamino)phenyl)-3,3′″-dihexyl-[2,2′:5′,2″:5″,2′″-quaterthiophene]-5-carbaldehyde (0.30 g, 0.39 mmol) from step 3 and 1-(2-ethylhexyl)-4-methyl-2,6-dioxo-1,2,5,6-tetrahydropyridine-3-carbonitrile (0.184 g, 0.70 mmol) were placed in a microwave reactor tube (10.0-20.0 ml) and to this mixture dichloromethane (12 ml) was added followed by pyridine (0.055 g). The mixture was heated at 75° C. for two hours in a microwave reactor (Initiator™ Biotage microwave reactor). The progress of reaction was followed by TLC which indicated the consumption of starting aldehyde. The dark blue reaction mixture was cooled, methanol (20 ml) was added and the product was collected by filtration and washed with hot methanol (3×30 ml). The crude material was purified by flash chromatography eluted with 100% CHCl₃ to afford 0.36 g (91.2%) of title product as shiny bluish-black solid.

HPLC (10% THF/ACN): 94%; M. Pt.: 153-158° C.; IR (neat, cm⁻¹) 3063, 2954, 2928, 2859, 2215, 1686, 1638, 1592, 1542, 1492, 1407, 1383, 1295, 1177, 822, 782, 696;

¹H NMR (400 MHz, CD₂Cl₂) δ 7.87 (s, 1H), 7.65 (s, 1H), 7.49-7.45 (m, 3H), 7.33-7.27 (m, 6H), 7.12-7.05 (m, 10H), 3.95-3.92 (m, 2H), 2.89 (t, 2H, J=7.92 Hz), 2.83 (t, 2H, J=7.92 Hz), 2.62 (s, 3H), 1.90-1.82 (m, 8H), 1.48-1.30 (m, 17H), 0.94-0.91 (m, 12H);

¹³NMR (200 MHz, CDCl₃) δ 163.19, 160.92, 158.02, 149.35, 148.28, 147.47, 147.37, 143.67, 142.33, 141.09, 140.56, 140.47, 136.93, 135.41, 134.77, 133.67, 129.31, 128.67, 127.73, 126.31, 126.13, 125.34, 124.95, 124.58, 124.30, 123.45, 123.19, 116.22, 115.06, 103.47, 43.93, 37.42, 31.65, 31.59, 30.47, 30.42, 29.88, 29.66, 29.32, 29.22, 29.18, 28.41, 23.86, 23.07, 22.59, 22.55, 18.85, 14.12, 14.07, 14.05, 10.58; MS/EI: for C₆₂H₆₇N₃O₂S₄ (m/z) 1014.1

### Compound Example 6

### Step 1. Synthesis of 1-(6-hydroxyhexyl)-4-methyl-2,6-dioxo-1,2,5,6-tetrahydropyridine-3-carbonitrile

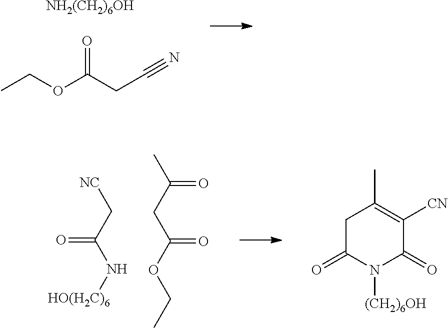

6-Amoino-1-hexanopl (11.72 g, 100.0 mmol) was placed in 250 ml round bottom flask under nitrogen and was cooled to 0° C. in an ice bath. Ethyl cyanoacetate was added slowly via dropping funnel over 20 minutes. Then the reaction mixture was stirred over night at room temperature. To the resulting pale yellow slurry was added a mixture of ethyl acetoacetate (13.0 g, 100.0 mmol) and piperidine (10 ml, 130.0 mmol). The reaction mixture was heated to 110° C. for 24 hours. The reaction mixture was then cooled and stirred for another day. The mixture was acidified to PH 1 using conc. HCl and the resulting slurry was stirred for an hour before collection by vacuum filtration. The solid was washed with dilute HCl and then with water. The pale pink solid was dried in vacuum oven @ 50° C. for 24 Hours to give 9.8 g (78%) of the title compound as pale beige solid. ¹H NMR showed that the compound existed as an enol in DMSO.

¹H NMR (200 MHz, DMSO) δ 5.67 (s, 1H), 4.71 (broad s, 1H), 3.86 (t, 2H J=7.3 Hz), 3.34 (t, 2H, J=6.1 Hz), 2.20 (s, 3H), 1.63-1.37 (m, 4H), 1.34-1.23 (m, 4H).

### Step 2. Synthesis of 5-((5′-((4-(diphenylamino)phenyl)-(2,2′-bithiophenyl-5-yl)methylene)-1-(6-hydroxyhexyl)-4-methyl-2,6-dioxo-1,2,5,6-tetrahydropyridine-3-carbonitrile

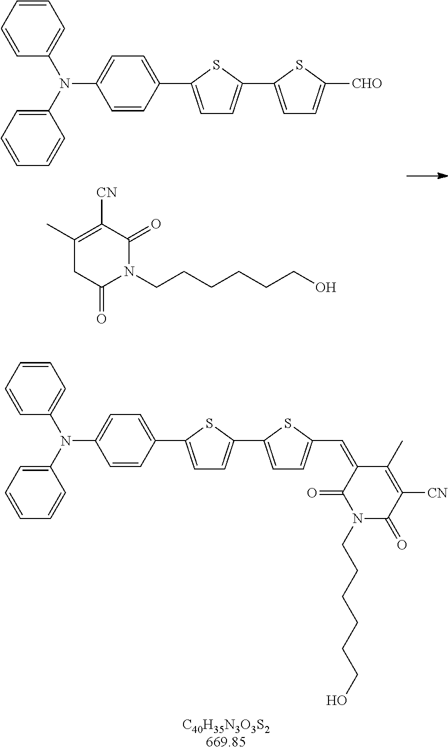 [SMILES: O=Cc1ccc(-c2ccc(-c3ccc(N(c4ccccc4)c4ccccc4)cc3)s2)s1 ; CC1=C(C#N)C(=O)N(CCCCCCO)C(=O)C1 ; CC1=C(C#N)C(=O)N(CCCCCCO)C(=O)/C1=C\c1ccc(-c2ccc(-c3ccc(N(c4ccccc4)c4ccccc4)cc3)s2)s1]

5′-[4-(diphenylamino)phenyl]-2,2′-bithiaphene-5-carbaldehyde (0.2900 g, 0.661 mmol) from Compound Example 2 and 1-(hydroxymethyl)-4-methyl-2,6-dioxo-1,2,5,6-tetrahydropyridine-3-carbonitrile (0.250 g, 1.00 mmol) from step 1 were placed in a microwave reactor tube (10.0-20.0 ml) and ethanol (12 ml) was added to this mixture. The mixture then heated 100° C. for 0.75 hours in a microwave reactor (Initiator™ Biotage microwave reactor). The progress of reaction was followed by TLC which indicated the consumption of starting aldehyde. The dark blue reaction mixture was cooled, isopropanol (20 ml) was added and the product was collected by filtration and washed with hot methanol (3×30 ml) to afford 0.320 g (79%) of the title compound (example 6) as deep blue solid.

HPLC (10% THF/ACN): 95%; M. Pt.: 253-259° C.; IR (neat, cm⁻¹) 3505 (bd), 2222, 1683, 1635, 1591, 1530, 1488, 1419, 1377, 1328, 1296, 1188, 1083, 697; ¹H NMR (400 MHz, CD₂Cl₂) δ 7.92 (s, 1H), 7.75 (d, 1H J=4.4 Hz), 7.43 (d, 1H J=4.4 Hz), 7.52-7.42 (m, 3H), 7.33-7.29 (m, 5H), 7.15-7.06 (m, 8H), 4.00 (t, 2H, J=7.4 Hz), 3.7-3.5 (m, 2H), 2.63, (s, 3H), 1.73-1.56 (m, 4H), 1.44-1.40 (m, 4H); ¹³C NMR (200 MHz, CDCl₃) δ 162.93, 160.66, 158.14, 154.82, 148.39, 148.14, 147.08, 144.05, 135.95, 134.15, 129.42, 128.69, 126.65, 124.96, 124.40, 123.64, 122.77, 115.65, 115.02, 103.19, 62.83, 40.14, 32.59, 27.66, 26.63, 25.28, 18.88; HRMS/EI: calcd for C₄₀H₃₅N₃O₃S₂ (m/z) 669.2114; found=669.2106. UV-Vis (CHCl₃ solution) λ max 598 nm (onset 725 nm); molar extinction coefficient (ε)=˜54865 M-¹ cm⁻¹ UV-Vis (CHCl₃ film) λ max 597 nm (onset 792 nm). Energy gap (ΔE)=1.56 eV HOMO (PESA): −5.40 eV; LUMO: −3.84 eV

### Compound Example 7

### Step 1. Synthesis of 6-(3-cyano-5-((5′-(4-(diphenylamino)phenyl)-[2,2′-bithiophen]-5-yl)methylene)-4-methyl-2,6-dioxo-5,6-dihydropyridin-1(2H)-yl)hexyl methacrylate

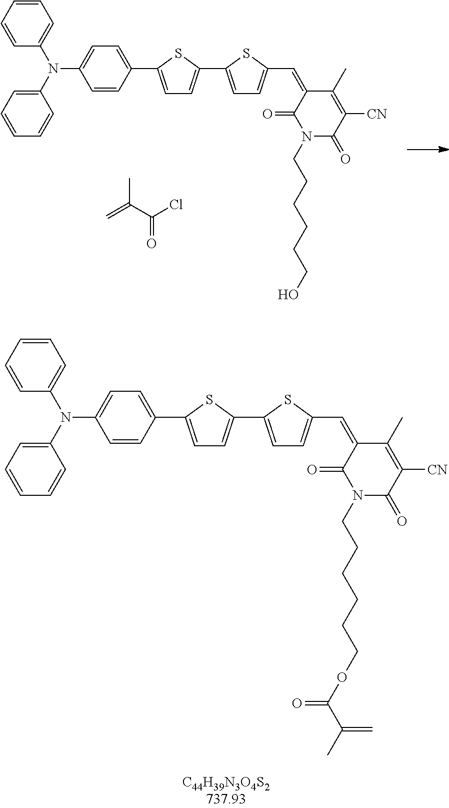

To a solution of 5-((5-(4-(diphenylamino)phenyl)-[2,2′-bithiophen]-5-yl)methylene)-1-(6-hydroxyhexyl)-4-methyl-2,6-dioxo-1,2,5,6-tetrahydropyridine-3-carbonitrile (2.0 g, 2.981 mmol) from Compound Example 6 in dry dichloromethane (40 ml), was added triehyl amine (0.410 ml, 3.57 mmol) at 0° C. The mixture stirred under nitrogen for 5 minutes and then methacryloyl chloride was added drop wise via dropping funnel. The reaction mixture was stirred at room temperature over night. The progress of the reaction was followed by TLC. The reaction mixture was transferred to separating funnel with the aid of dichloromethane, washed with dilute HCl, water and finally with brine. The organic layer was dried on anhydrous magnesium sulphate, filtered and the solvent was removed under reduced pressure to get crude solid which was purified through column chromatography on silica eluted with 50% pet. ether (60-80° C.)/chloroform→100% chloroform to afford 1.2 g of the titled compound (example 7) as deep blue solid.

HPLC (10% THF/ACN): 96%; M. Pt.: 166-171° C.; IR (neat, cm⁻¹) 3063, 2952, 2852, 2219, 1716, 1677, 1629, 1592, 1545, 1530, 1488, 1426, 1377, 1295, 1190, 1162, 1086, 1058, 784, 698; ¹H NMR (400 MHz, CDCl₃) δ 7.87 (s, 1H), 7.69 (d, 1H J=4.3 Hz), 7.54 (d, 1H J=4.0 Hz), 7.50-7.46 (m, 2H), 7.37 (d, 1H J=4.3 Hz), 7.31-7.27 (m, 4H), 7.24 (d, 1H J=4.0 Hz), 7.15-7.04 (m, 8H), 6.08-6.04 (m, 1H), 5.53-5.52 (m, 1H), 4.14 (t, 2H J=6.3 Hz), 4.06-4.0 (m, 2H), 2.62 (s, 3H), 1.93 (s, 3H), 1.74-1.66 (m, 4H). 1.44-1.43 (m, 4H); ¹³C NMR (400 MHz, CDCl₃) δ 167.54, 162.94, 160.65, 158.17, 154.85, 148.41, 148.17, 147.11, 144.08, 136.49, 135.98, 134.17, 129.45, 129.33, 128.71, 126.68, 126.65, 125.23, 124.99, 124.43, 123.68, 123.65, 122.79, 115.67, 115.04, 103.22, 64.70, 40.19, 28.53, 27.67, 26.64, 25.74, 18.92, 18.34; HRMS/EI: calcd for C₄₄H₃₉N₃O₄S₂ (m/z) 737.2377; found=737.2388.

### Variations

Further compounds within general formula I can be prepared through the selection of appropriate starting materials. For example, in the preparation of compounds of type C as per steps 3 and 8 above, compounds of type A can be synthesised or purchased with the appropriate groups R₁, R₂ and Ar and with linker L if desired. Compounds of type C can also be prepared with the appropriate groups R₃, R₄ and R₉ and with the appropriate number of thiophene units (T) (of n in number). From those starting materials, the appropriate formyl precursor containing the selected groups R₁, R₂, R₃, R₄ and n is prepared. If substitution at R₅ is desired, an iodo-ketone can be synthesised. Reaction of the formyl/keto precursor with an appropriate compound of type D is then undertaken as per steps 6 and 9 above to produce the desired target compound.

Suitable compounds of type D can be prepared through the selection of appropriate starting materials. For example, in the preparation of the compounds of type D as per steps 4 and 5 above, the ethyl acetoacetate and the amine can be synthesised or purchased with the appropriate R₆ and R₇ groups present respectively.

### Bilayer Solar Cell

A bilayer organic solar cell (1) of one embodiment of the invention is illustrated in FIG. 1. The bilayer organic solar cell comprises a transparent layer of indium tin oxide as the anode (2) supported on a transparent thin film support (3), and a cathode (4) in the form of a metal cathode, opposite. Between the anode and cathode are layers of the compound of formula I (5) as the electron donor material (or p-conductor), and an electron acceptor material (6) (or n-conductor) such as fullerene. The device may contain multiple layers, and the term “bilayer” should be interpreted as encompassing 2 or more layered devices. The device may be in the form of a single cell, or multiple cells connected in parallel and/or series. The device typically further comprises positive and negative terminals (not illustrated) for connection to an energy storage device or other electrical component(s) or circuit(s).

### Bulk Heterojunction Solar Cell

A bulk heterojunction organic solar cell (7) of one embodiment of the invention is illustrated in FIG. 2. In this figure, elements that are common to the bilayer solar cell (1) of FIG. 1 are referred to using the same numerals. The bulk heterojunction organic solar cell (7) comprises a transparent layer of indium tin oxide as the anode (2) supported on a transparent thin film support (3), and a cathode (4) in the form of a metal cathode, opposite. Between the anode and cathode is an active layer comprising a blend of electron acceptor material (6) (or n-conductor) such as fullerene, and the compound of formula I (5) as the electron donor (or p-conductor). The concentration of each component (5) and (6) gradually increases when approaching to the corresponding electrode. The device may be in the form of a single cell, or multiple cells connected in parallel and/or series. The device typically further comprises positive and negative terminals (not illustrated) for connection to an energy storage device or other electrical component(s) or circuit(s).

### Dye Sensitised Solar Cell

A dye sensitised solar cell (8) of one embodiment of the invention is illustrated in FIG. 3. In this Figure, elements that are common to the bilayer solar cell (1) of FIG. 1 are referred to using the same numerals. The dye sensitised solar cell comprises a transparent layer of indium tin oxide as the anode (2) supported on a transparent thin film support (3). A layer of particulate titanium dioxide (9) of an average particle size of 20 nm is located on the surface of the anode (2), which is an n-type inorganic semiconductor material and acts as an electron acceptor material. The titanium dioxide layer (9) is coated on its surface with the compound of formula I, acting as the sensitiser, or electron donor material. This is represented schematically by an area marked with the numeral (5) in FIG. 1, but in reality would be a thin coating on the particles. This is applied by any suitable technique, such as by dissolving in a solvent, and contacting with the titanium dioxide layer, to load the sensitiser onto the surface. A cathode (4) in the form of a metal cathode is placed above the layer of sensitiser (5), and an electrolyte (10) filled in the space between the sensitiser (5) and the cathode (4), contacting the two materials. The electrolyte is of any suitable type, and in the illustrated embodiment is typically the iodine/triodide red/ox couple. Other electrolytes maybe ionic liquid or solid or polymeric electrolytes. The edges of the device are sealed to encase the electrolyte (10) between the anode (2) and cathode (4). The device may be in the form of a single cell, or multiple cells connected in parallel and/or series. The device typically further comprises positive and negative terminals (not illustrated) for connection to an energy storage device or other electrical component(s) or circuit(s).

### Test Work on Photovoltaic Devices Containing Compound Example 2.

### Apparatus

Indium tin oxide (ITO) coated glass with a sheet resistance of 15Ω/square was purchased from Kintek. Polyethylenedioxythiophene/polystyrenesulfonate (“PEDOT/PSS”) (Baytron P Al 4083) was purchased from HC Starck. PCBM and C60 were purchased from Nano-C. Calcium pellets and 2,9-dimethyl-4,7-diphenyl-1,10-phenanthroline (BCP) were purchased from Aldrich. Aluminium pellets (99.999%) were purchased from KJ Lesker.

UV-ozone cleaning of ITO substrates was performed using a Novascan PDS-UVT, UV/ozone cleaner with the platform set to maximum height, the intensity of the lamp is greater than 36 mW/cm² at a distance of 100 cm. At ambient conditions the ozone output of the UV cleaner is greater than 50 ppm.

Aqueous solutions of PEDOT/PSS were deposited in air using a Laurell WS-400B-6NPP Lite single wafer spin processor. Organic blends were deposited inside a glovebox using an SCS G3P Spincoater. Film thicknesses were determined using a Dektak 6M Profilometer. Vacuum depositions were carried out using an Edwards 501 evaporator inside a glovebox. Samples were placed on a shadow mask in a tray with a source to substrate distance of approximately 25 cm. The area defined by the shadow mask gave device areas of 0.1 cm². Deposition rates and film thicknesses were measured using a calibrated quartz thickness monitor inside the vacuum chamber. C60 was evaporated from a boron nitride crucible wrapped in a tungsten filament. BCP was evaporated from a baffled tantalum boat. Ca and Al (3 pellets) were evaporated from separate, open tungsten boats.

### Methods

ITO coated glass was cleaned by standing in a stirred solution of 5% (v/v) Deconex 12PA detergent at 90° C. for 20 mins. The ITO was successively sonicated for 10 minutes each in distilled water, acetone and iso-propanol. The substrates were then exposed to a UV-ozone clean (at room temperature) for 10 minutes. The PEDOT/PSS solution was diluted by 50% in methanol, filtered (0.2 μm RC filter) and deposited by spin coating at 5000 rpm for 60 sec to give a 38 nm layer. The PEDOT/PSS layer was then annealed on a hotplate in the glovebox at 140° C. for 10 minutes. Where used, solutions of the organic blends were deposited onto the PEDOT/PSS layer by spin coating inside a glovebox (H₂O and O₂ levels both <1 ppm). Spinning conditions and film thicknesses were optimised for each blend. The devices were transferred (without exposure to air) to a vacuum evaporator in an adjacent glovebox. Where used, single layers of the organic materials were deposited sequentially by thermal evaporation at pressures below 2×10⁻⁶ mbar. Where used, a layer of Ca was deposited by thermal evaporation at pressures below 2×10⁻⁶ mbar. For all devices a layer of Al was deposited by thermal evaporation at pressures below 2×10⁻⁶ mbar. Where noted, the devices were then annealed on a hotplate in the glovebox.

A small amount of silver paint (Silver Print II, GC electronics, Part no.: 22-023) was deposited onto the connection points of the electrodes. Completed devices were encapsulated with glass and a UV-cured epoxy (Lens Bond type J-91) by exposing to 254 nm UV-light inside a glovebox (H₂O and O₂ levels both <1 ppm) for 10 minutes. Electrical connections were made using alligator clips.

The cells were tested with an Oriel solar simulator fitted with a 1000 W Xe lamp filtered to give an output of 100 mW/cm² at AM 1.5. The lamp was calibrated using a standard, filtered Si cell from Peccell limited (The output of the lamp was adjusted to give a JSC of 0.605 mA). The estimated mismatch factor of the lamp is 0.95. Values were not corrected for this mismatch.

The Incident Photon Collection Efficiency (IPCE) data was collected using an Oriel 150 W Xe lamp coupled to a monochromator and an optical fibre. The output of the optical fibre was focussed to give a beam that was contained within the area of the device. The IPCE was calibrated with a standard, unfiltered Si cell.

For both the solar simulator and the IPCE measurements devices were operated using a Keithley 2400 Sourcemeter controlled by Labview Software.

The measurements on the solar simulator gave the cell efficiency under AM 1.5 illumination. The measurements on the IPCE setup gave them cell efficiency at individual wavelengths. For block co-polymers the IPCE spectrum will demonstrate contributions to the overall efficiency from both components of the polymer.

### RESULTS

### Device Example 1

### Compound Example 2 was used in a blend device with the fullerene derivative [6,6]-phenyl-C61-butyric acid methyl ester (PCBM) as the second material

Device structure: ITO/PEDOT:PSS (38 nm)/Compound Example 2:PCBM (1:1) (40 nm)/Ca (20 nm)/Al (100 nm).

A 1 cm³ solution of Compound Example 2 (10 mg) and PCBM (10 mg) in chlorobenzene was prepared by stirring for 30 mins. The solution was filtered (0.2 μm RC filter) and spin coated at 1000 rpm for 60 second. Vacuum deposition of the Ca (20 nm) and Al (100 nm) layers were done in the glove box.

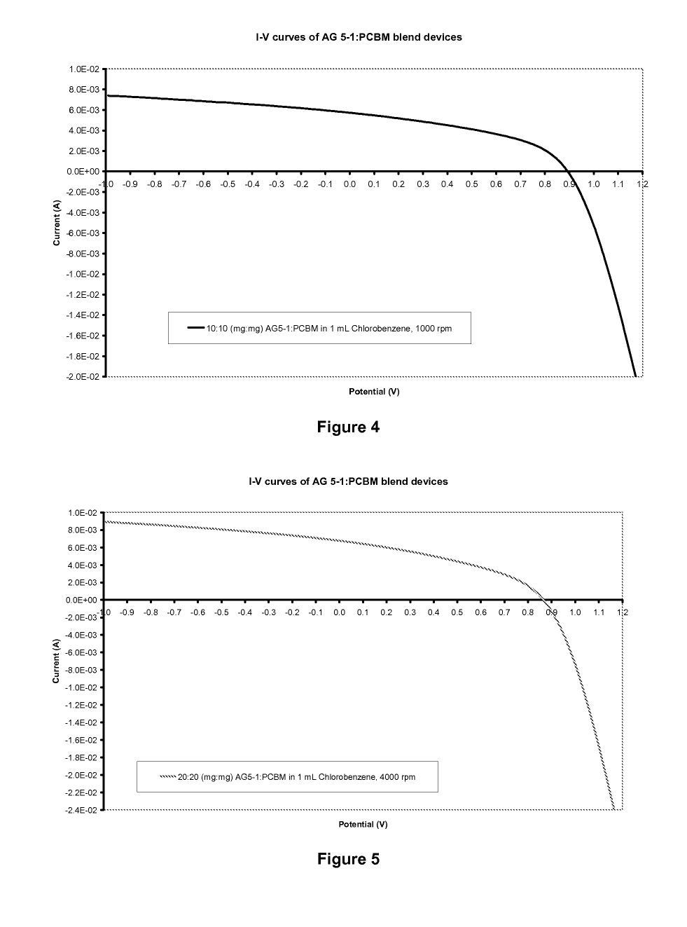

The I-V curve for the device is shown in FIG. 4. The device parameters were V_{OC}=901 mV, I_{SC}=5.73 mA/cm², FF=43%, PCE=2.2%.

### Device Example 2

Compound Example 2 was Used in a Blend Device with PCBM.

Device tructure: ITO/PEDOT:PSS (38 nm)/Compound Example 2:PCBM (1:1)/Ca (20 nm)/Al (100 nm).

A 1 cm³ solution of Compound Example 2 (20 mg) and PCBM (20 mg) in chlorobenzene was prepared by stirring for 30 mins. The solution was filtered (0.2 μm RC filter) and spin coated at 4000 rpm for 60 second. Vacuum deposition of the Ca (20 nm) and Al (100 nm) layers were done in the glove box.

The I-V curve for the device is shown in FIG. 5. The device parameters were V_{OC}=871 mV, I_{SC}=6.77 mA/cm², FF=38%, PCE=2.25%.

### Compound Example 2 Devices

| | | Voltage | Current | | | |
|---|---|---|---|---|---|---|
| | Spin speed | o/c | s/c | Fill | Efficiency | Thickness |
| Sample | rpm | mV | mA/cm2 | Factor | % | nm |
| 10:10 Compound Example | 1000 | 901.35 | 5.73 | 0.43 | 2.2 | ~38 |
| 2:PCBM in CB | | | | | | |
| 20:20 Compound Example | 4000 | 871.5 | 6.77 | 0.38 | 2.25 | |
| 2:PCBM in CB | | | | | | |
| 20:20 Compound Example | 4000 | 891.4 | 7.32 | 0.36 | 2.34 | |
| 2:PC(70)BM in CB | | | | | | |
| 15:15 Compound Example | 2000 | 901.35 | 5.95 | 0.35 | 1.88 | |
| 2:PC(70)BM in CB | | | | | | |
| 10:10 Compound Example | 4000 | 892.45 | 5.5 | 0.31 | 1.54 | <100 |
| 2:PCBM in CF | | | | | | |
| 20:40 Compound Example | 4000 | 870.65 | 5.7 | 0.33 | 1.62 | |
| 2:PCBM in CB | | | | | | |
| 10:40 Compound Example | 2000 | 811.75 | 5.43 | 0.32 | 1.41 | |
| 2:PCBM in CB | | | | | | |

Annealing:
10:10 Compound Example 2:PCBM in CB

| | |
|---|---|
| no annealing: | 2.2% |
| annealing at 150° C.: | 0.65% |
| annealing at 210° C.: | 0.0% |

10:40 Compound Example 2:PCBM in CB

| | |
|---|---|
| no annealing: | 1.41% |
| annealing at 150° C.: | 1.01% |
| annealing at 210° C.: | 0.0% |

| | Spin | Voltage | Current | | Max | | | |
|---|---|---|---|---|---|---|---|---|
| | speed | o/c | s/c | Fill | Power | V(pmax) | Efficiency | Thickness |
| Active layer | rpm | mV | mA/cm2 | Factor | mW/cm2 | mV | % | nm |
| 10:10 Compound Example | 1000 | 880.6 | 6.29 | 0.44 | 2.45 | 621.9 | 2.45 | ~38 nm |
| 2:PCBM in 1 mL CB | | | | | | | | |
| 10:10 Compound Example | 4000 | 892.45 | 5.5 | 0.31 | 1.54 | 513.95 | 1.54 | <100 nm |
| 2:PCBM in 1 mL CF | | | | | | | | |
| 10:40 Compound Example | 4000 | 792.85 | 2.46 | 0.34 | 0.665 | 494.00 | 0.67 | >155 nm |
| 2:PCBM in 1 mL CF | | | | | | | | |
| 10:10 Compound Example | 1000 | 901.35 | 5.73 | 0.43 | 2.2 | 642.55 | 2.2 | ~38 nm |
| 2:PCBM in1 mL CB | | | | | | | | |
| 10:40 Compound Example | 2000 | 811.75 | 5.43 | 0.32 | 1.411 | 483.25 | 1.41 | |
| 2:PCBM in 1 mL CB | | | | | | | | |
| 20:20 Compound Example | 4000 | 871.5 | 6.77 | 0.38 | 2.25 | 572.85 | 2.25 | |
| 2:PCBM in 1 mL CB | | | | | | | | |
| 20:40 Compound Example | 4000 | 870.65 | 5.7 | 0.33 | 1.62 | 522.4 | 1.62 | |
| 2:PCBM in 1 mL CB | | | | | | | | |
| 20:20 Compound Example | 4000 | 891.4 | 7.32 | 0.36 | 2.34 | 543.0 | 2.34 | |
| 2:PC(70)BM in 1 mL CB | | | | | | | | |
| 20:20 Compound Example | 3000 | 872.5 | 2.18 | 0.27 | 0.505 | 444.2 | 0.51 | |
| 2:PC(70)BM in 1 mL CF | | | | | | | | |
| 15:15 Compound Example | 2000 | 901.35 | 5.95 | 0.35 | 1.88 | 552.95 | 1.88 | |
| 2:PC(70)BM in 1 mL CB | | | | | | | | |
| 15:15 Compound Example | 2000 | 572.85 | 2.25 | 0.42 | 545.7 | 363.8 | 0.55 | |
| 2:PC(70)BM in 1 mL oDCB | | | | | | | | |

Device Structure:
ITO coated glass
PEDOT:PSS: filtered before spin coating using 0.2 μm syringe filter, spin cast at 5000 rpm for 60 second and annealed at 140° C. for 10 minutes in the glove box
Active layer: as it shows in the table (x:x Compound Example 2:PCBM means 10 mg Compound Example 2 and 10 mg PCBM was solved in 1 mL solvent)
CB=Chlorobenzene
CF=Chloroform
oDCB=ortho-Dichlorobenzene
20 nm Ca and 100 nm Al was vacuum deposited on the top of the active layer in the glove box
Sealing: UV-curable epoxy glue was used to place a covering glass slide on top of the evaporated metal electrodes to protect device areas from air exposure. Al and ITO contacts were thinly covered with silver-paint in the glove box.

### Device Example 3

Compound Example 3 was Used in a Blend Device with PCBM.
Device structure: ITO/PEDOT:PSS (38 nm)/Compound Example 3:PCBM (1:1)/Ca (20 nm)/Al (100 nm).

A 1 cm³ solution of Compound Example 3 (20 mg) and PCBM (20 mg) in chlorobenzene was prepared by stirring at 50° C. for 60 mins. The solution was filtered (0.2 μm RC filter) and spin coated at 4000 rpm for 60 second. Vacuum deposition of the Ca (20 nm) and Al (100 nm) layers were done in the glove box.

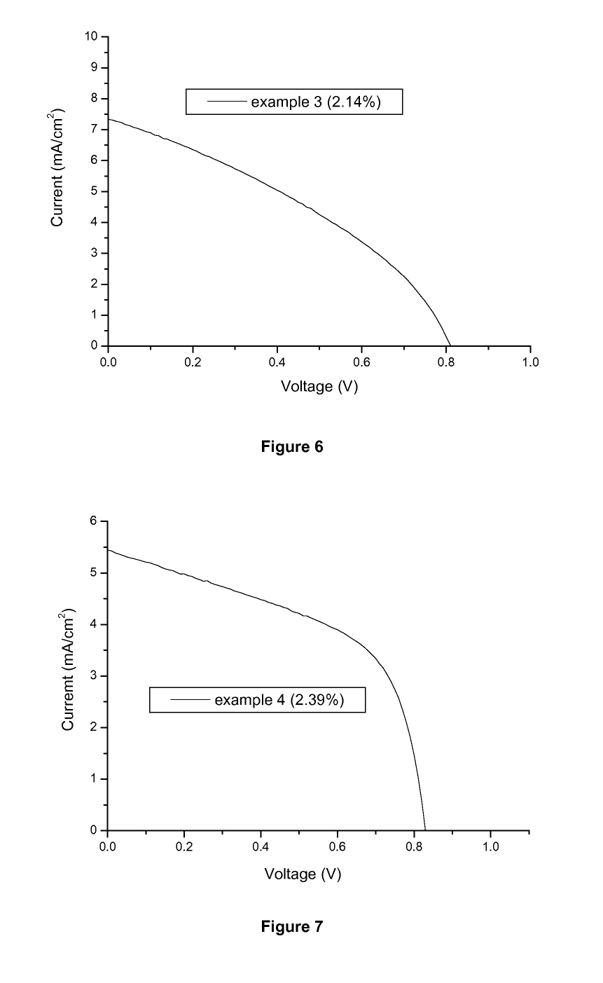

The I-V curve for the device is shown in FIG. 6. The device parameters were V_{OC}=810 mV, I_{SC}=7.33 mA/cm², FF=36%, PCE=2.14%.

### Compound Example 3 Devices

| | Spin | Voltage | Current | | Effi- | Thick- |
|---|---|---|---|---|---|---|
| | speed | o/c | s/c | Fill | ciency | ness |
| Sample | rpm | mV | mA/cm² | Factor | % | nm |
| 20:20 Compound | 4000 | 810 | 7.33 | 0.36 | 2.14 | 62 |
| Example 3:PCBM | | | | | | |
| in CB | | | | | | |
| 8:32 Compound | 4000 | 740 | 5.14 | 0.33 | 1.27 | 53 |
| Example 3:PCBM | | | | | | |
| in CB | | | | | | |

Device Structure:
ITO coated glass
PEDOT:PSS: filtered before spin coating using 0.2 μm syringe filter, spin cast at 5000 rpm for 20 second and annealed at 150° C. for 10 minutes in the glove box
Active layer: as it shows in the table (x:x Compound example 3:PCBM means, for instance, 20 mg of compound example 3 and 20 mg of PCBM was dissolved in 1 mL solvent)

The formed film from each blend concentration is annealed at 120° C. for 10 min before metal electrode deposition
CB=Chlorobenzene
CF=Chloroform
oDCB=ortho-Dichlorobenzene
20 nm Ca and 100 nm Al was vacuum deposited on the top of the active layer in the glove box.

### Device Example 4

Compound Example 4 was Used in a Blend Device with PCBM.

Device structure: ITO/PEDOT:PSS (38 nm)/Compound Example 4:PCBM (1:4)/Ca (20 nm)/Al (100 nm).

A 1 cm³ solution of Compound Example 4 (8 mg) and PCBM (32 mg) in chlorobenzene was prepared by stirring at 50° C. for 60 mins. The solution was filtered (0.2 μm RC filter) and spin coated at 4000 rpm for 60 second. Vacuum deposition of the Ca (20 nm) and Al (100 nm) layers were done in the glove box.

The I-V curve for the device is shown in FIG. 7. The device parameters were V_{OC}=830 mV, I_{SC}=5.44 mA/cm², FF=52.9%, PCE=2.39%.

### Compound Example 4 Devices

| | Spin | | Voltage | Current | | | |
|---|---|---|---|---|---|---|---|
| | speed | Anneal | o/c | s/c | Fill | Efficiency | Thickness |
| Sample | rpm | T | mV | mA/cm2 | Factor | % | nm |
| 8:32 Compound Example | 4000 | 120 | 830 | 5.44 | 0.53 | 2.39 | 62 |
| 4:PCBM in CB | | | | | | | |
| 20:20 Compound Example | 4000 | 140 | 830 | 5.36 | 0.45 | 2.0 | |
| 4:PCBM in CB | | | | | | | |

Device Structure:
ITO coated glass
PEDOT:PSS: filtered before spin coating using 0.2 μm syringe filter, spin cast at 5000 rpm for 20 second and annealed at 150° C. for 10 minutes in the glove box
Active layer: as it shows in the table (x:x Compound example 4:PCBM means, for instance, 8 mg of compound example 4 and 32 mg of PCBM was dissolved in 1 mL solvent)

The formed film is annealed at 120° C. or 140° C. for 10 min before metal electrode deposition
CB=Chlorobenzene
CF=Chloroform
oDCB=ortho-Dichlorobenzene
20 nm Ca and 100 nm Al was vacuum deposited on the top of the active layer in the glove box.

### Device Example 5

Compound Example 5 was Used in a Blend Device with PCBM.

Device structure: ITO/PEDOT:PSS (38 nm)/Compound Example 5:PCBM (1:3)/Ca (20 nm)/Al (100 nm).

A 1 cm³ solution of Compound Example 5 (10 mg) and PCBM (30 mg) in chlorobenzene was prepared by stirring at 50° C. for 60 mins. The solution was filtered (0.2 μm RC filter) and spin coated at 4000 rpm for 60 second. Vacuum deposition of the Ca (20 nm) and Al (100 nm) layers were done in the glove box.

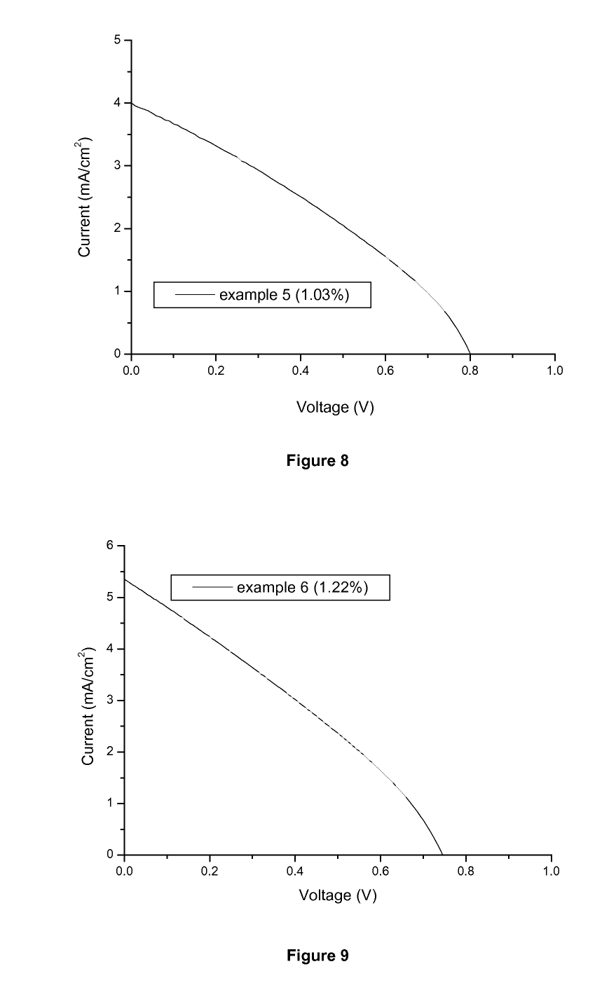

The I-V curve for the device is shown in FIG. 8. The device parameters were V_{OC}=800 mV, I_{SC}=3.70 mA/cm², FF=32.5%, PCE=0.96%.

### Compound Example 5 Devices

| | Spin | | Voltage | Current | | | |
|---|---|---|---|---|---|---|---|
| | speed | Annea [symbol] | o/c | s/c | Fill | Efficiency | Thickness |
| Sample | rpm | Tem. | mV | mA/cm2 | Factor | % | nm |
| 10:10 Compound Example | 4000 | 120 | 800 | 2.80 | 0.34 | 0.76 | 54 |
| 5:PCBM in CB | | | | | | | |
| 10:10 Compound Example | 5000 | 120 | 750 | 2.73 | 0.33 | 0.67 | 53 |
| 5:PCBM in CB | | | | | | | |
| 10:30 Compound Example | 4000 | 120 | 800 | 3.70 | 0.33 | 0.96 | |
| 5:PCBM in CB | | | | | | | |
| 10:30 Compound Example | 4000 | 140 | 0.78 | 3.56 | 0.33 | 0.93 | |
| 5:PCBM in CB | | | | | | | |
| 10:30 Compound Example | 4000 | 160 | 0.67 | 3.43 | 0.30 | 0.70 | |
| 5:PCBM in CB | | | | | | | |
| 8:32 Compound Example | 4000 | 120 | 0.80 | 4.00 | 0.32 | 1.03 | |
| 5:PCBM in CB | | | | | | | |
| 8:32 Compound Example | 4000 | 140 | 0.78 | 3.64 | 0.33 | 0.93 | |
| 5:PCBM in CB | | | | | | | |
| 8:32 Compound Example | 3000 | 140 | 0.79 | 3.59 | 0.32 | 0.89 | |
| 5:PCBM in CB | | | | | | | |

[symbol]

indicates data missing or illegible when filed

Device Structure:
ITO coated glass
PEDOT:PSS: filtered before spin coating using 0.2 μm syringe filter, spin cast at 5000 rpm for 20 second and annealed at 150° C. for 10 minutes in the glove box
Active layer: as it shows in the table (x:x Compound example 5:PCBM means, for instance, 10 mg of compound example 5 and 10 mg of PCBM was dissolved in 1 mL solvent)

The films were formed from different blend concentration and the formed film is annealed at variable temperatures for 10 min before metal electrode deposition
CB=Chlorobenzene
CF=Chloroform
oDCB=ortho-Dichlorobenzene
20 nm Ca and 100 nm Ai was vacuum deposited on the top of the active.

### Device Example 6

Compound Example 6 was Used in a Blend Device with PCBM.

Device structure: ITO/PEDOT:PSS (38 nm)/Compound Example 6:PCBM (1:3)/Ca (20 nm)/Al (100 nm).

Compound Example 6 is not very soluble in CB but fully soluble in CF at room temperature under the same concentration. A 1 cm³ solution of Compound Example 6 (10 mg) and PCBM (10 mg) in chloroform was prepared by stirring at room temperature for 60 mins. The solution was filtered (0.2 μm RC filter) and spin coated at 3000 rpm for 60 second. After annealing at 110° C. for 10 min, vacuum deposition of the Ca (20 nm) and Al (100 nm) layers were done in the glove box.

The I-V curve for the device is shown in FIG. 9. The device parameters were V_{OC}=750 mV, I_{SC}=5.35 mA/cm², FF=30%, PCE=1.22%.

### Compound Example 6 Devices

| | Spin | Annea [symbol] | Voltage | Current | Fill | | |
|---|---|---|---|---|---|---|---|
| Sample | speed | Tem. | o/c | s/c | Factor | Efficiency | Thickness |
| 15:15 Compound Example | 4000 | 110 | 65 | 0.97 | 0.25 | 0.02 | 45 |
| 6:PCBM in CB | | | | | | | |
| 15:15 Compound Example | 4000 | No | 625 | 2.62 | 0.39 | 0.64 | 45 |
| 6:PCBM in CB | | anneal | | | | | |
| 15:15 Compound Example | 3000 | 110 | 20 | 1.95 | 0.19 | 0.01 | 76 |
| 6:PCBM in CB | | | | | | | |
| 10:10 Compound Example | 1000 | 110 | 650 | 4.43 | 0.37 | 1.07 | 43 |
| 6:PCBM in CB | | | | | | | |
| 10:10 Compound Example | 1000 | No | 620 | 3.94 | -/39 | 0.95 | 43 |
| 6:PCBM in CB | | anneal | | | | | |
| 10:10 Compound Example | 2000 | 110 | 620 | 2.18 | 0.41 | 0.55 | |
| 6:PCBM in CB | | | | | | | |
| 10:10 Compound Example | 3000 | 110 | 750 | 5.35 | 0.30 | 1.22 | 135 |
| 6:PCBM in CF | | | | | | | |

[symbol]

indicates data missing or illegible when filed

Device Structure:
ITO coated glass
PEDOT:PSS: filtered before spin coating using 0.2 μm syringe filter, spin cast at 5000 rpm for 20 second and annealed at 150° C. for 10 minutes in the glove box
Active layer: as it shows in the table (x:x Compound example 6:PCBM means, for instance, 10 mg of compound example 6 and 10 mg of PCBM was dissolved in 1 mL solvent)

The films were formed at different concentration under variable spin speed and the formed film is annealed at 110° C. for 10 min or without annealing as required before metal electrode deposition
CB=Chlorobenzene
CF=Chloroform
oDCB=ortho-Dichlorobenzene
20 nm Ca and 100 nm Al was vacuum deposited on the top of the active layer in the glove box.

It will be understood to persons skilled in the art of the invention that many modifications may be made without departing from the spirit and scope of the invention.

It is to be understood that, if any prior art publication is referred to herein, such reference does not constitute an admission that the publication forms a part of the common general knowledge in the art, in Australia or any other country.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A compound of formula I: 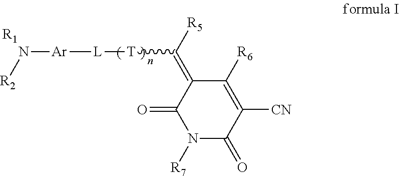
wherein:
R₁ and R₂ are independently selected from the group consisting of optionally substituted C₁-C₂₀ alkyl, optionally substituted C₃-C₈ cycloalkyl, optionally substituted aromatic, and optionally substituted heteroaromatic groups or R₁ and R₂ together with the nitrogen atom to which they are attached comprise an optionally substituted saturated or unsaturated ring which may optionally contain further heteroatoms selected from the group consisting of O, N and S, and may optionally be further fused to one or more other rings;
Ar is selected from the group consisting of optionally substituted aromatic and optionally substituted heteroaromatic groups;
L is a linker which is a direct bond or is selected from the group consisting of optionally substituted C₂ alkenylene and optionally substituted C₂ alkynylene;
T is independently selected from the group consisting of: 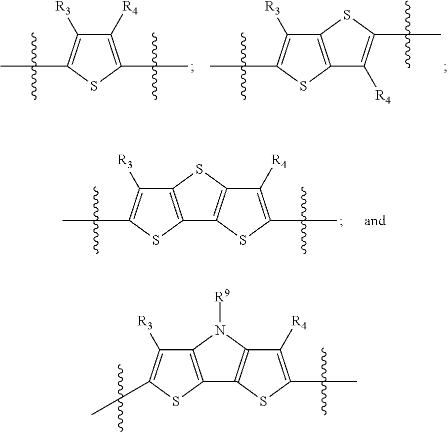
R₃, R₄ and R₉ are independently selected from the group consisting of hydrogen, optionally substituted C₁-C₁₀ alkyl, optionally substituted C₃-C₈ cycloalkyl and optionally substituted C₁-C₁₀ alkoxy groups, or a pair of groups selected from R₃, R₄ and R₉ may together with the carbon atoms to which they are attached comprise an optionally substituted saturated or unsaturated ring which may optionally contain one or more heteroatoms selected from the group consisting of O, N and S, and may optionally be further fused to one or more other rings;
R₅ is selected from the group consisting of hydrogen, optionally substituted C₁-C₈ alkyl, optionally substituted C₃-C₈ cycloalkyl, and optionally substituted aromatic groups;
R₆ is selected from the group consisting of optionally substituted C₁-C₈ alkyl, optionally substituted C₁-C₈ perfluorinated alkyl, optionally substituted C₃-C₈ cycloalkyl, optionally substituted aromatic, and optionally substituted heteroaromatic groups;
R₇ is selected from the group consisting of optionally substituted C₁-C₃₀ alkyl wherein one or more carbon atoms of the alkyl are optionally replaced with one or more of O, S, NR₈, carbonyl or thiocarbonyl; optionally substituted C₃-C₈ cycloalkyl; optionally substituted C₂-C₁₂ alkenyl wherein one or more carbon atoms of the alkenyl are optionally replaced with one or more of O, S, NR₈, carbonyl or thiocarbonyl; optionally substituted C₂-C₈ alkynyl wherein one or more carbon atoms of the alkynyl are optionally replaced with one or more of O, S, NR₈, carbonyl or thiocarbonyl; optionally substituted C₃-C₁₂ alkoxy; optionally substituted aromatic; and optionally substituted heteroaromatic groups; wherein R₈ is hydrogen or R₆; and
n is an integer of 1 to 10.

2. The compound of claim 1, wherein R₁ and R₂ are independently selected from the group consisting of phenyl, substituted phenyl, fluorenyl, and substituted fluorenyl.

3. The compound of claim 1 or 2, wherein Ar is phenyl, fluorenyl, dialkylfluoroenyl or thiophenyl.

4. The compound of any one of claims 1 to 3, wherein L is a direct bond.

5. The compound of any one of claims 1 to 4, wherein R₃ is hydrogen.

6. The compound of any one of claims 1 to 5, wherein R₄ is hydrogen.

7. The compound of any one of claims 1 to 4, wherein R₃ and R₄ are both hydrogen.

8. The compound of any one of claims 1 to 4, wherein one of R₃ and R₄ is hydrogen, and the other of R₃ and R₄ is optionally substituted C₁-C₁₀ alkyl.

9. The compound of any one of claims 1 to 8, wherein R₉ is hydrogen.

10. The compound of any one of claims 1 to 8, wherein R₉ is optionally substituted C₁-C₁₀ alkyl.

11. The compound of any one of claims 1 to 4, wherein R₃ and R₄ are hydrogen and R₉ is optionally substituted C₁-C₁₀alkyl.

12. The compound of any one of claims 1 to 11, wherein R₅ is hydrogen.

13. The compound of any one of claims 1 to 12, wherein R₆ is selected from the group consisting of methyl, ethyl and CF₃.

14. The compound of any one of claims 1 to 13, wherein R₇ is selected from the group consisting of optionally substituted C₁-C₃₀ alkyl wherein the optional substituents are selected from the group consisting of hydroxyl, carboxylic acid, methacryloxy, acryloxy, hydroxyalkyleneoxy, 2-bromo-2-methylpropanoate, trimethylsilyl and silyl ether; aromatic groups substituted with a carboxylic acid group, and optionally substituted 5- or 6-heteroaromatic groups containing one or more heteroatoms selected from the group consisting of O, N and S.

15. A photovoltaic device comprising:
a first electrode,
a second electrode,
an active material in electrical contact with the first and second electrodes, the active material comprising:
(i) a compound of formula I as defined in any one of claims 1 to 14; and
(ii) a second material which is a charge accepting material, wherein the device generates an electrical potential upon the absorption of photons.

16. The photovoltaic device of claim 15, wherein the first electrode is an anode, and the second electrode is a cathode.

17. The photovoltaic device of claim 16, wherein the anode is a metal oxide anode, and the cathode is a metal or metal alloy cathode.

18. The photovoltaic device of any one of claims 15 to 17, wherein the second material is an electron acceptor material.

19. The photovoltaic device of any one of claims 15 to 18, wherein the second material is an n-type inorganic semiconductor material.

20. The photovoltaic device of claim 15, wherein the first electrode is a cathode, the second electrode is an anode, and the second material is an electron donating material.

21. The photovoltaic device of claim 20, wherein the second material is a p-type inorganic semiconductor material.

22. The photovoltaic device of any one of claims 15 to 21, further comprising an electrolyte in contact with the compound of formula I and the second electrode.

23. A dye sensitised solar cell comprising:
an anode,
a cathode,
a charge accepting material on one electrode,
a compound of formula I as defined in any one of claims 1 to 14, in contact with the charge accepting material; and
a charge transport material in contact with the compound of formula I and the other electrode.

24. The solar cell of claim 23, wherein the anode is a metal oxide anode and the cathode is a metal or metal alloy cathode.

25. The solar cell of any one of claims 23 to 24, wherein the charge accepting material is an electron acceptor material.

26. The solar cell of claim 25, wherein the electron acceptor material is an n-type inorganic semiconductor material.

27. The solar cell of claim 31, wherein the cathode is a metal oxide cathode, the anode is a metal or metal alloy anode, and the charge accepting material is a p-type inorganic semiconductor material.

28. A process for the preparation of a compound of formula I as defined in claim 1, comprising reacting compound C: 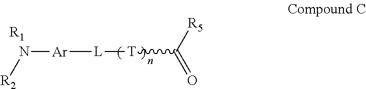
wherein R₁, R₂, R₅, Ar, L, T and n are as defined in claim 1,
with compound D: 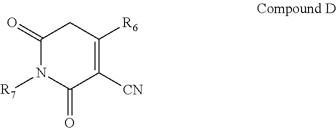
wherein R₆ and R₇ are as defined in claim 1.
